(19) United States
(12) Patent Application Publication
(10) Pub. No.: US 2026/0204418 A1
(43) Pub. Date: Jul. 16, 2026

(54) SYSTEM, METHOD, AND COMPUTER READABLE MEDIUM FOR CGM-BASED OGTT REPLACEMENT DIAGNOSIS TEST

(71) Applicant: UNIVERSITY OF VIRGINIA PATENT FOUNDATION, Charlottesville, VA (US)

(72) Inventors: Leon S. FARHI, Charlottesville, VA (US); Eslam Montaser Roushdi ALI, Charlottesville, VA (US); Boris P. KOVATCHEV, Charlottesville, VA (US)

(73) Assignee: UNIVERSITY OF VIRGINIA PATENT FOUNDATION, Charlottesville, VA (US)

(21) Appl. No.: 18/870,862

(22) PCT Filed: Jun. 2, 2023
(86) PCT No.: PCT/US2023/024301

§ 371 Date: Dec. 2, 2024

Related U.S. Application Data
(60) Provisional application No. 63/348,315, filed on Jun. 2, 2022.

(51) Int. Cl. G16H 50/30; A61B 5/145; G16H 20/17

(52) U.S. Cl. CPC G16H 50/30; A61B 5/14532; G16H 20/17

(57) ABSTRACT

Embodiments relate to a database management system for efficient physiological diagnosis of a specified physiological disorder. The system includes a physical data store containing glucose measurement data and a representation for a classification of the glucose measurement data. The glucose measurement data is associated with controlled glycemic-response consumption activity, the representation being indication that a glucose measurement trace is associated of a specified physiological disorder or is indicative of the specified physiological disorder, or is a surrogate of an existing metabolic test, e.g. OGTT. A processor receives a new glucose measurement possibly associated with controlled glycemic-response consumption activity, and classifies the newly received glucose measurement trace with the representation based either on a disease- or test-specific classifier or based on a matched similarity metric, and ascribes a clinical recommendation or assessment based on the representation of the classified newly received glucose measurement trace.

### CROSS-REFERENCE TO RELATED APPLICATIONS

This patent application is related to and claims the benefit of priority of U.S. Provisional Application No. 63/348,315, filed on Jun. 2, 2022, the entire contents of which is incorporated by reference.

### STATEMENT REGARDING FEDERALLY SPONSORED RESEARCH AND DEVELOPMENT

This invention was made with government support under Grant No. DK106907 awarded by the National Institutes of Health. The government has certain rights in the invention.

### FIELD

Embodiments can relate to a database management system for efficient physiological diagnosis of a specified physiological disorder.

### BACKGROUND INFORMATION

Early screening for identifying individuals at increased diabetes risk can reduce the rates of complications at diagnosis, improve the post-diagnosis quality of glycemic control, potentially slow the progression, reduce future poor health outcomes and diabetes-related complications. Predicting the risk for developing diabetes however poses a significant challenge and many efforts have been devoted to develop simple screening tests, including metabolic and genetic tests. Of significant clinical relevance is these tests to be cheap, easy to administer, provide as much information as possible, and present minimal burden to the individual. Ideally, as proposed in this application, they should be self-administered without the need for a visit to a clinical facility or use of a clinical lab. They should also estimate not only the life-long risk for an individual to develop diabetes, but should be also able to assess the state of progression and the short-term risk for imminent diagnosis. For example, screening for genetic diabetes risk (both type 1 and type 2) can be performed as an at-home test. However, this test cannot account for the level of progression towards development of full-blown diabetes. As another example, the Oral Glucose Tolerance Test (OGTT) can be used for diagnosis of diabetes and estimating the level of glucose tolerance impairment. However, an OGTT requires a visit to a health care facility and use of a medical lab. The present invention is an alternative to the mentioned above OGTT that can be self-administered and does not require use of a clinical lab.

Until the present invention, how to integrate screening for, for example, T1D risk into practice remained unclear. The current data for progression to T1D points to risk influenced largely by immunological and genetic factors, with unknown environmental triggers. Its identification and quantification are challenging and, as a result, screening for T1D risk is not currently recommended as standard practice. We echo the statement in the RFA: “ . . . major limitations preventing this integration is the utilization of research-grade genetics or autoantibody testing platforms by these programs. Integration of these programs into clinical care will require that these assays be adapted to platforms commonly used in hospital or commercial medical laboratories. In addition, as demonstrated by the recent COVID-19 pandemic, offering at-home or commercially available testing platforms has provided an important avenue for patients to get screened for disease and disease risk.”

Taking again T1D as an example, the detection of the immunological risk for T1D is based upon presence of islet autoantibodies; however, who and when to test for appearance of autoantibodies has been both a concern and a limitation. Autoantibody testing requires a visit to a laboratory or a hospital/clinic for sample collection and processing. This testing is typically limited to research subjects and/or individuals with family history of T1D. In contrast, 95% of those who develop T1D have no family history of the disease yet could be identified as having “high T1D risk” if tested. As the rate of DKA at T1D onset is ~40% in those who are not monitored, the detection of those at high risk without a family history of T1D is critical. One approach for initial screening is to use genetics (which does not change over time). T1D genetic risk assessment can be performed as a self-administered, at-home test, but requires resources and does not account for the immunologic risk. In addition, the relationship between immunologic risk (through presence of islet autoantibodies) and genetic risk is not clearly established; thus, the art in this technological field can benefit from development of a unified, self-administered, at-home test that can simultaneously and reliably predict the immunological and genetic risk for T1D.

There are several clinical tests to diagnose diabetes (https://diabetes.org/diabetes/a1c/diagnosis). These include blood tests for HbA1C, fasting plasma glucose, 2-hour OGTT, random plasma glucose tests, etc. In addition, diagnosis of type 1 diabetes requires follow-up tests for islet autoantibodies and/or ketones in the urine. Notably, these tests require a visit to a health care facility and use of a clinical laboratory. Screening for diabetes in healthy individuals can be performed by an islet auto-antibody tests (T1D) or genetic tests (T1D and T2D) at home, with some recent attempts to use Continuous Glucose Monitor devices (CGM) as an alternative or as a complementary test.

Screening for gestational diabetes mellitus (GDM). Women who are at average risk of GDM (overweight and with family history of diabetes) are currently recommended an OGTT between 24 and 28 gestational weeks as the method of GDM diagnosis. The proposed here OGTT replacement can reduce the burden of the OGTT data collection.

Published data on CGM use for assessment of diabetes risk. Several studies employed CGM not only in people with diabetes but also in obese individuals and in individuals at different stages of prediabetes. Studies have suggested that CGM can be used for detecting early hyperglycemia in children with multiple autoantibodies, and for predicting progression to diabetes in antibody positive (Ab+) children. It has been suggested that “CGM should be included in the ongoing monitoring of high-risk children (Ab+)”. In addition, in T2D, CGM was able to detect impaired glycemia earlier than other standard biomarkers used for diagnosis and classification. Recently, a one-week CGM test has been investigated for its ability to identify individuals at higher risk for rapid progression to Stage 3 T1D (full-blown disease) and had identified several CGM-derived metrics of hyperglycemia associated with progression to Stage 3.

### SUMMARY

Embodiments can relate to a database management system for efficient physiological diagnosis of a specified physiological disorder. The system can include a physical data store containing glucose measurement data and a representation for at least one classification of the glucose measurement data. The glucose measurement data can be associated with at least one controlled glycemic-response consumption activity. The representation can be an indication that a glucose measurement trace from the glucose measurement data is associated with a specified physiological disorder or is indicative of the specified physiological disorder. The system can include a processor and computer memory configured with instructions stored thereon that when executed will cause the processor to receive a new glucose measurement possibly associated with controlled glycemic-response consumption activity. The instructions can cause the processor to search the physical data store by comparing a newly received glucose measurement trace to the at least one classification using a similarity metric. The instructions can cause the processor to classify the newly received glucose measurement trace with the representation based on either a prespecified feature-based classifier or by using a matched similarity metric in response to the comparing. The instructions can cause the processor to ascribe a clinical recommendation or assessment based on the representation of the classified newly received glucose measurement trace.

Embodiments can relate to a method for managing a database for efficient physiological diagnosis of a specified physiological disorder. The method can involve receiving a glucose measurement possibly associated with controlled glycemic-response consumption activity. The method can involve searching a physical data store by comparing a newly received glucose measurement trace to at least one classification using a similarity metric. The physical data store can contain glucose measurement data and a representation for the at least one classification of the glucose measurement data. The glucose measurement data can be associated with at least one controlled glycemic-response consumption activity. The representation can be an indication that a glucose measurement trace from the glucose measurement data is associated with a specified physiological disorder or is indicative of the specified physiological disorder. The method can involve classifying the newly received glucose measurement trace with the representation based on a matched similarity metric in response to the comparing. The method can involve ascribing a clinical recommendation or assessment based on the representation of the classified newly received glucose measurement trace.

### BRIEF DESCRIPTION OF THE DRAWINGS

Other features and advantages of the present disclosure will become more apparent upon reading the following detailed description in conjunction with the accompanying drawings, wherein like elements are designated by like numerals, and wherein:

FIG. 1 is an exemplary system that can be used for processing glucose data by efficient glucose database management;

FIG. 2 represents three different panels of CGM traces aggregated to create a single ambulatory glucose profile (AGP) as a visual display in different autoantibodies (Ab) groups (i.e., Negative, 1 Ab, and 2≥Ab);

FIG. 3 shows a characterization of CGM data through different glycemic features in different scenarios;

FIG. 4 shows characterization of CGM data through different glycemic features in different scenarios;

FIG. 5 shows the distribution of different BPs in 8 clusters based on three different categories of AB;

FIG. 6 summarizes the accuracy of different models through receiver-operating characteristic (ROC) curves, sensitivity, and specificity as measures to test a classifier's performance;

FIG. 7 shows variable importance;

FIG. 8 shows an exemplary high-level functional block diagram for embodiments of the system;

FIG. 9 shows an exemplary network system in which embodiments of the system and method can be implemented;

FIG. 10 shows an exemplary a block diagram that illustrates a system including a computer system and the associated Internet connection upon which an embodiment may be implemented;

FIG. 11 shows an exemplary system in which one or more embodiments of the system and methods can be implemented using a network, or portions of a network, or computers; and

FIG. 12 shows an exemplary block diagram illustrating an example of a machine upon which one or more aspects of embodiments of the system and method can be implemented.

### DETAILED DESCRIPTION

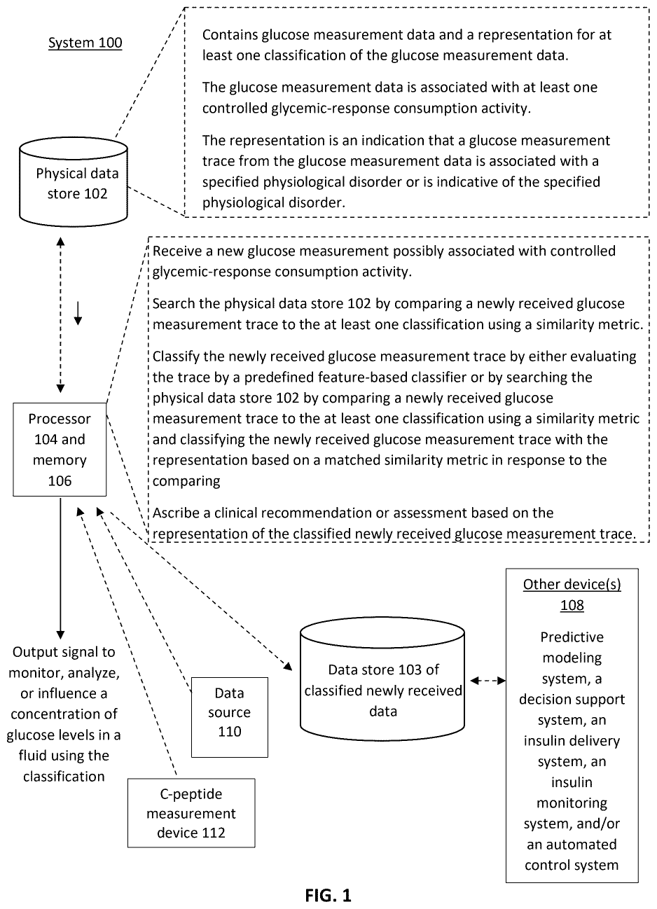

Referring to FIG. 1, embodiments can relate to a database management system 100 for efficient physiological diagnosis of a specified physiological disorder. While exemplary embodiments describe systems and methods for diagnosis of type 1 diabetes, it is understood that the systems and methods can be applied to other metabolic disorders (e.g., type 2 diabetes, metabolic syndrome, gestational diabetes etc.). The database management system 100 can include a physical data store 102. The physical data store 102 can contain glucose measurement data and a representation for at least one classification of the glucose measurement data. The glucose measurement data can be associated with at least one controlled glycemic-response consumption activity. As will be explained herein, the glucose measurement data can be obtained from plural individuals while each individual engages in a controlled glycemic-response consumption activity (e.g., glycemic-response consumption activity that is predetermined). For instance, the controlled glycemic-response consumption activity can be consumption of a specific amount of a specified energy drink at specified times. This energy drink can elicit a glycemic-response, indicative of a specified physiological disorder. As a non-limiting example, consumption of an energy drink can elicit a glycemic response indicative of presence of islet autoantibodies which in turn is a marker of the insulin beta cells in the pancreas of an individual being or becoming damaged—i.e., indicative of the individual being or becoming diabetic.

The glucose measurement data, being associated with controlled glycemic-response consumption activity, can capture this glycemic-response. For instance, if glucose measurements are taken when individuals engage in controlled glycemic-response consumption activity, the glucose measurement data can be associated with the glycemic-response. A priori knowledge of whether these individuals have or are at risk of the specified physiological disorder can be used to develop a trained data set. The trained data set can be used to develop a classifier model, which can be used to classify new glucose measurement data of a patient. Thus, a processor 104 implementing the classifier model can effectively and efficiently classify new glucose measurement data of a patient to determine if the data suggests that the patient has/does not have the specified disorder, is a high/low risk of developing the specified disorder, etc. As can be appreciated from the above, the representation can be an indication that a glucose measurement trace from the glucose measurement data in the physical data store 102 is associated with a specified physiological disorder or is indicative of the specified physiological disorder.

The database management system 100 can include a processor 104 and computer memory 106 configured with instructions stored thereon that when executed will cause the processor to receive a new glucose measurement possibly associated with controlled glycemic-response consumption activity. While it is contemplated for the individual to engage controlled glycemic-response consumption activity for the new glucose measurement, this data may or may not be associated with controlled glycemic-response consumption activity.

As noted herein, the instructions can cause the processor 104 to implement a classifier model. In this regard, the instructions can cause the processor 104 to search the physical data store 102 by comparing a newly received glucose measurement trace to the at least one classification using a similarity metric. The instructions can cause the processor 104 to classify the newly received glucose measurement trace with the representation based on a matched similarity metric in response to the comparing.

The instructions can cause the processor 104 to ascribe a clinical recommendation or assessment (e.g., diagnosis, risk score, disease progression level, estimate for a specific metabolic test outcome, disease-specific test outcome, treatment, etc.) based on the representation of the classified newly received glucose measurement trace. The treatment can be a command signal, a modification signal, a recommendation, etc. for an insulin dose, a bolus dose, an exercise routine, a meal consumption routine, a medication routine, etc.

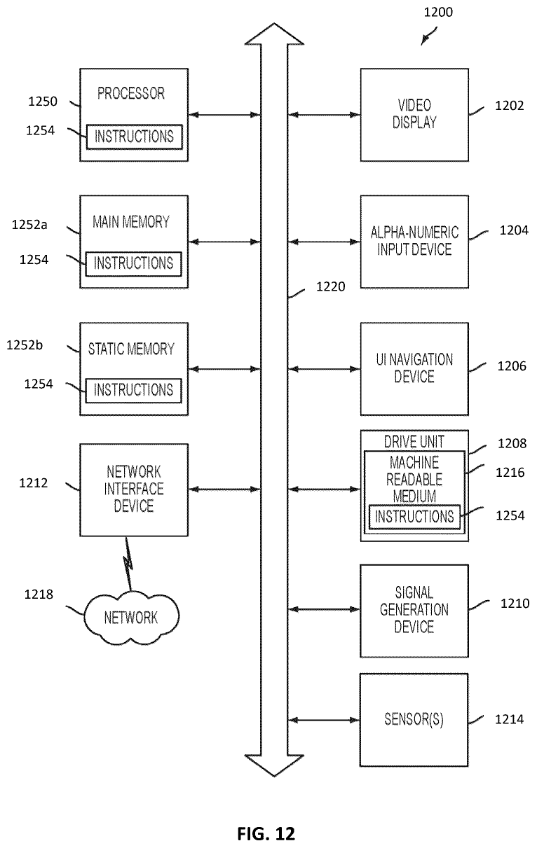

The processor 104, as well as any of the processors disclosed herein, can be part of or in communication with a machine 1200 (logic, one or more components, circuits (e.g., modules), or mechanisms-see FIG. 12). The processor 104 can be hardware (e.g., processor, integrated circuit, central processing unit, microprocessor, core processor, computer device, etc.), firmware, software, etc. configured to perform operations by execution of instructions embodied in algorithms, data processing program logic, artificial intelligence programming, automated reasoning programming, etc. It should be noted that use of processors herein includes any one or combination of a Graphics Processing Unit (GPU), a Field Programmable Gate Array (FPGA), a Central Processing Unit (CPU), etc. The processor 104 can include one or more processing modules. A processing module can be a software or firmware operating module configured to implement any of the method steps disclosed herein. The processing module can be embodied as software and stored in memory, the memory being operatively associated with the processor 104. A processing module can be embodied as a web application, a desktop application, a console application, etc. Exemplary embodiments of the processor 104 and the machine 1200 are discussed later.

The processor 104 can include or be associated with memory 106 or a computer or machine readable medium 1216. As discussed in more detail later, the computer or machine readable medium 1216 can include memory 1252a, 1252b. Any of the memory 106 discussed herein can be computer readable memory configured to store data. The memory 106 can include a volatile or non-volatile, transitory or non-transitory memory, and be embodied as an in-memory, an active memory, a cloud memory, etc. Embodiments of the memory 106 can include a processor module and other circuitry to allow for the transfer of data to and from the memory 106, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc.

The computer or machine readable medium 1216 can be configured to store one or more instructions thereon. The instructions can be in the form of algorithms, program logic, etc. that cause the processor 104 to build and/or implement one or more of the classification models disclosed herein.

The processor 104 can be in communication with other processor(s) of other device(s) 108 (e.g., a predictive modeling system, a decision support system, an insulin delivery system, an insulin recommendation system, a glycemic state or insulin monitoring system, a glucose or insulin management system, an automated control system, etc.) configured to use the classification or the representation as input. Any of those other device(s) 108 can include any of the exemplary processors disclosed herein. Any of the processors can have transceivers or other communication devices/circuitry to facilitate transmission and reception of wireless signals. Any of the processors can include an Application Programming Interface (API) as a software intermediary that allows two applications to talk to each other. Use of an API can allow software of the processor 104 of the system 100 to communicate with software of the processor of the other device(s) 108.

Any of the transmissions between processors/devices/systems/modules can be a push, operation, a pull operation, or a combination of both. Any of the transmissions can be direct transmission between two components or transmission via an intermediary. An intermediary may be memory, database, data store, etc. for example. For instance, data from one processor may be transmitted to a database for storage before being transmitted to another processor. As another example, data may be transmitted to an intermediary processor or processing module to process the data, format the data, encode the data, etc. before being transmitted to another processor. Data transmission between components can be continuously, periodically, at some other predetermined schedule, as-demanded by control signals, based on a condition being met per algorithmic function, etc.

### Exemplary System/Method for Developing a Database for Classifying Glucose Data

As can be appreciated form the present disclosure, embodiment can relate to systems and methods for developing a database to classify glucose data. The system can include a processor, which may or may not be the same processor 104 used to classify new glucose measurements. The system can include computer memory having instructions stored thereon that when executed will cause the processor to implement any of the method steps disclosed herein. The instructions can cause the processor to receive glucose data (e.g., glucose measurement data). The glucose data can include one or more glucose measurements. The glucose measurements can be a time series of measurements that represent a glucose level profile (e.g., a pattern, a behavior, a trend, etc.). The glucose data can be historical, current, and/or real-time data. The glucose data is received by the processor. This can be done continuously, periodically, or at some other predetermined schedule. The glucose data can be pulled by the processor from a data source 110 and/or pushed from the data source 110 to the processor. The data source 110 can be a device that generates glucose measurements (e.g., a glucose monitor/sensor, a continuous glucose monitor/sensor, an assay device, etc.) or a data store 103 (e.g., database) that stores glucose data. The glucose measurements can be of a fluid, such as interstitial fluid, etc. The processor can store the glucose data in transient or persistent memory for later processing or process the glucose profile data as it is being received. For instance, the processor can receive glucose profile data and aggregate the glucose data in storage. The aggregation can be based on the type of data, what the data represents, the time of receiving the data, the time the data was generated, etc., which can be embodied in metadata for example. The processor can include one or more classifier models to classify the glucose data. Any of the classifier models can use on or more artificial intelligence models to classify the glucose data. It is contemplated for the glucose data to be collected for one or more individuals. It is further contemplated for the glucose data to be collected while in the one or more individuals engages in controlled glycemic-response consumption activity (e.g., the glucose data is collected before, during, and/or after one or more individuals engages in controlled glycemic-response consumption activity). The controlled glycemic-response consumption activity can be an energy drink, for example. By doing this, the glycemic response to the consumption activity is captured in the glucose data. One or more training sets can be developed and used to train a classification model. The one or more individuals can be individual(s) that may have low risk of the specified physiological disorder and not diagnosed with the specified physiological disorder, individual(s) that have high risk of the specified physiological disorder and not diagnosed with the specified physiological disorder, and/or individual(s) diagnosed with the specified physiological disorder. The trained data set can train the classifier model to classify glucose data based on whether those individuals exhibited one of the categories above. The classifier model can then be implemented on a processor 104 of the database management system for efficient physiological diagnosis of new glucose data obtained from a new patient.

In developing the database, the glucose data can include glucose measurements from one or more individuals. This robust data set can allow the model to be used to determine glycemic trends, predict glycemic states, use multivariate analyses regarding conditions and factors (e.g., eating behavior, exercise behavior, medical condition, age, gender, race, heart rate, respiratory rate, blood oxygen saturation, etc.) that cause or relate to a glycemic state, etc. For instance, multivariable modeling techniques can be used to determine which conditions or factors statistically contribute to a change glycemic state, a change in risk of hypo- or hyper-glycemia, etc., which can also be used to estimate the probabilities of the same. The multivariable modeling technique can include one or more of logistic regression with or without cubic splines, random forest, xgboost, support vector machines, nearest neighbor, artificial neural networks, and/or long short-term memory (LS™), multivariate analysis of variance (MANOVA), multivariate analysis of covariance (MANCOVA), principal components analysis (PCA), canonical correlation analysis, redundancy analysis (RDA), correspondence analysis (CA), canonical correspondence analysis (CCA), multidimensional scaling, discriminant analysis, linear discriminant analysis (LDA), clustering systems, recursive adaptive partitioning, vector autoregression, principal response curves analysis (PRC), etc.

### Exemplary Systems/Methods for Classifying Glucose Data

As noted above, embodiments can relate to a database management system 100 for efficient physiological diagnosis of a specified physiological disorder. The database management system 100 can include a physical data store 102. The physical data store 102 can contain glucose measurement data and a representation for at least one classification of the glucose measurement data. The glucose measurement data can be associated with at least one controlled glycemic-response consumption activity. The representation can be an indication that a glucose measurement trace from the glucose measurement data is associated with presence or absence of islet autoantibodies. The database management system 100 can include a processor 104 and computer memory 106 configured with instructions stored thereon that when executed will cause the processor 104 to receive a new glucose measurement possibly associated with controlled glycemic-response consumption activity. The instructions can cause the processor 104 to search the physical data store 102 by comparing a newly received glucose measurement trace to the at least one classification using a similarity metric. The instructions can cause the processor 104 to classify the newly received glucose measurement trace with the representation based on a matched similarity metric in response to the comparing. The instructions can cause the processor 104 to ascribe a clinical recommendation or assessment (e.g., diagnosis, risk score, disease progression level, estimate for a specific metabolic test outcome, disease-specific test outcome, treatment, etc.) based on the representation of the classified newly received glucose measurement trace. The treatment can be a command signal, a modification signal, a recommendation, etc. for an insulin dose, a bolus dose, an exercise routine, a meal consumption routine, a medication routine, etc.

It is contemplated for the new glucose measurement received by the processor 104 to be from a single individual so as to classify the glucose data of that individual by comparing the individual's glucose data to classified glucose measurement data in the database; however, the new glucose measurement can be of one or more individuals. It is contemplated for the new glucose measurement to be recent data (e.g., data collected in real-time or within the past 24 hours), but the new glucose measurement can be historical, current, and/or real-time data. The instructions can cause the processor 104 to classify the new glucose measurement, or a portion thereof, either by using a prespecified classifier or by comparing new glucose measurement to a pre-classified embodiment in the database. The instructions can cause the processor 104 to store the classification or representation of the new glucose measurement in a data store 103 that is in communication with another device(s) 108 (e.g., one or more of a predictive modeling system, a decision support system, an insulin delivery system, an insulin monitoring system, an automated control system, etc.) configured to use the classification or representation as input. In addition, or in the alternative, the instructions can cause the processor 104 to transmit the classification or representation of glucose profile data to another device(s) 108 (e.g., one or more of a decision support system, an insulin delivery system, an insulin monitoring system, etc.) configured to use the classification or representation as input. In addition, or in the alternative, the instructions can cause the processor 104 to monitor, analyze, and/or influence a concentration of glucose levels in a fluid using the classification or representation.

The instructions can cause the processor 104 to classify the new glucose measurement by comparing the new glucose measurement to classified glucose measurement data in the database. For instance, new glucose measurement can include a glucose measurement trace (e.g., a newly received glucose measurement trace) that is a compilation of plural new glucose measurements. This trace can be compared to one or more glucose measurement traces from the glucose measurement data in the physical data store 102 that has been classified. A new trace that exactly, approximately, or similarly matches with a stored trace can be classified as having an exact, approximate, or similar classification. There can be one or more exact, approximate, or similar classification matches.

In some embodiments, the representation for the one or classification allows the processor 104 to stop its search of the physical data store 102 or narrow its search within the physical data store 102, thereby providing an even more efficient means to search the physical data store 102, classify data, and ascribe a clinical recommendation or assessment.

The treatment recommendation can include initiating, modifying, or forgoing administration of synthetic insulin based on the representation. If the classification of the new glucose measurement is of a representation of glucose measurement data associated with presence of an autoantibody that is indicative of the specified physiological disorder, then the recommendation can be forgoing administration. As another example, if the classification of the new glucose measurement is of a representation of glucose measurement data associated with absence of an autoantibody that is a representation of glucose measurement data associated with presence of an autoantibody that is indicative of the specified physiological disorder, then the recommendation can be initiating or modifying the short- or long-term administration schedule. The representation can be an estimation that a stored glucose measurement trace is classified as autoantibody positive or autoantibody negative. This estimation can be a statistic, a probability, a predictive analytic, a score, a weighted sore, etc. Thus, the recommendation, in the absence or presence of the autoantibody, can be to modify administration. If the autoantibody is associated with diabetes as the specific physiological disorder, then the administration can be of synthetic insulin. The treatment recommendation need not be limited to administration of medication. For instance, the treatment recommendation can be an insulin dose, a bolus dose, an exercise routine, a meal consumption routine, a medication routine, a increased glucose measurement routine, a recommendation to perform a specific medical diagnostic test, etc.

The controlled glycemic-response consumption activity can include consumption of an energy drink in lieu of a meal. It is contemplated for the controlled glycemic-response consumption activity to be controlled in the sense that its amount, time of consumption, frequency of consumption, etc. is predetermined. It is also contemplated for the controlled glycemic-response consumption activity to not be influenced or affected by other factors (e.g., consumption of other meals, consumption of other beverages, engaging in exercise, etc.). Thus, the controlled glycemic-response consumption activity can include consumption of one or more energy drinks in lieu of a meal. As will be explained herein, the gathering of glucose measurement data for the physical data store 102 and the gathering of the new glucose measurement can be done for a predetermined period of time (e.g., seven days). Thus, the controlled glycemic-response consumption activity can be in lieu of one or mor meals for one or more days within the predetermined period of time.

In some embodiments, the physical data store 102 contains glucose measurement data from one or more of: a) individuals that have low risk of the specified physiological disorder and not diagnosed with the specified physiological disorder; b) individuals that have high risk of the specified physiological disorder and not diagnosed with the specified physiological disorder; or c) individuals diagnosed with the specified physiological disorder. As noted therein, the classified glucose measurement data is associated with controlled glycemic-response consumption activity and is based on a trained data set based on priori knowledge of the individuals falling within one or more of the categories above.

In some embodiments, the newly received glucose measurement trace is from: a) an individual that has low risk of the specified physiological disorder and not diagnosed with the specified physiological disorder; b) an individual that has high risk of the specified physiological disorder and not diagnosed with the specified physiological disorder; or c) an individual diagnosed with the specified physiological disorder. The new glucose measurement from the patient that is to be classified can be an individual falling within one of the categories listed above. Thus, the system 100 and method disclosed herein can be used to test whether an individual has the specified physiological disorder, has a low risk of developing the specified physiological disorder, has a high risk of developing the specified physiological disorder, or has the specified physiological disorder.

Exemplary embodiments disclosed herein discuss and illustrate a system 100 and method for use with the specified physiological disorder being diabetes. In this regard, the autoantibody can be an islet antibody. However, other specified physiological disorders can be used and other autoantibodies can be used.

As noted herein, the representation can be an estimate that a stored glucose measurement trace is classified as autoantibody positive or autoantibody negative. The similarity metric can be a score that is a probability that the newly received glucose measurement trace is classified as autoantibody positive or autoantibody negative. For instance, the stored glucose measurement traces (the classified traces in the physical data store 102) having the best similarity metric can be used to classify the new glucose measurement trace. The similarity metric can be a numerical value falling within a range of value (e.g., from 0-1). A similarity metric of 0 can indicate a match, whereas a similarity metric of 1 can indicate a mismatch with a gradation of degree of matching between 0 and 1. Alternatively, a similarity metric of 1 can indicate a match, whereas a similarity metric of 0 can indicate a mismatch with a gradation of degree of matching between 1 and 0. Other similarity metric schemes can be used.

In some embodiments, the system 100 can include a data source (e.g., glucose measurement device 110) configured to generate the glucose measurement for the newly received glucose measurement trace. The glucose measurement device 110 can be in communication with the processor 104 or in communication with a data store 103 that is in communication with the processor 104.

It is contemplated for the glucose measurement data contained in the physical data store 102 to be continuous glucose measurement data; however, it need not be. It is further contemplated for the glucose measurements of the newly received glucose measurement trace to be continuous glucose measurement data; however, it need note be. Thus, a continuous glucose monitor can be used as a glucose measurement device 110 for generating the glucose measurement data for the physical data store 102. Similarly, a continuous glucose monitor can be used as a glucose measurement device 110 for generating the new glucose measurement to be classified by the processor 104 of the system 100.

It is further contemplated for the glucose measurement trace of the glucose measurement data contained in the physical data store 102 to be a compilation of glucose measurements taken for seven days, and the newly received glucose measurement trace to be a compilation of glucose measurements taken for seven days. For instance, a glucose measurement trace and/or a newly received glucose measurement trace can be a compilation of glucose measurements taken for a predetermined time period. Each trace can be, for example, a compilation of glucose measurements taken every 5 minutes for 24 hours a day (e.g., a continuous glucose measurement) for seven days. A trace can be a time series compilation of the glucose measurements over that predetermined time period.

The instructions can cause the processor 104 to store the classification of the newly received glucose measurement trace in a data store 103 that is in communication with another device(s) 108 (e.g., one or more of a predictive modeling system, a decision support system, an insulin delivery system, an insulin monitoring system, an automated control system configured to use the classification or representation as input, etc.). In addition, or in the alternative, the instructions can cause the processor 104 to transmit the classification of the newly received glucose measurement trace to another device(s) 108 (e.g., one or more of a predictive modeling system, a decision support system, an insulin delivery system, an insulin monitoring system, an automated control system configured to use the classification or representation as input, etc.). In addition, or in the alternative, the instructions can cause the processor 104 to monitor, analyze, or influence a concentration of glucose levels in a fluid using the classification of the newly received glucose measurement trace.

The classification of the glucose measurement data contained in the physical data store 102 can be based on one or more classifier models. Any of the classifier models can use on or more artificial intelligence models. Any of the classifier models can use a linear discriminant analysis (LDA) technique, a linear support vector machine (SVM) technique, a logistic regression (LR) technique, or a K-nearest neighbors (KNN) technique, etc. For instance, the one or more classifier models can be a model stored on memory associated with a processor that causes the processor to analyze, processes, and/or manipulate glucose measurement data and store the same in the physical data store 102. The classifier model can also generate the representation for at least one classification of the glucose measurement data. As a non-limiting example, the classifier model can generate or extract one or more glucose measurement metric from the glucose measurement data. The one or more glucose measurement metric can include: a mean glucose (MG) value; a percent of time in range glucose value; a coefficient of variation (CV) glucose value; a standard deviation (SD) glucose value; a glucose range; a low blood glucose index (LBGI) for hypoglycemia; a high blood glucose index (HBGI) for hypoerglycemia; an average daily risk range (ADRR) for hypo- and hyperglycemia; and/or an overnight incremental area under the curve (IAUC) value. One or more of these glucose measurement metrics can be used by the one or more classifier models. For instance, the one or more of these glucose measurement metrics can be used to determine whether, to what degree, what percentage, etc. glucose measurements in a trace fall within a time in range. This can be done to generate a percent of time in range glucose value.

The percent of time in range glucose value can be based on glucose measurements falling within one or more time ranges. The one or more time ranges can include: percentage of time glucose measurements are >180 mg/dL (T180); percentage of time glucose measurements are >160 mg/dL (T160); percentage of time glucose measurements are >140 mg/dL (T140); and percentage of time glucose measurements are <70 mg/dL (T70); and percentage of time glucose measurements are <54 mg/dl (T54).

In some embodiments, the physical data store 102 can include c-peptide data associated with the controlled glycemic-response consumption activity. In addition, the classification of the glucose measurement data can be further based on the c-peptide data. In this regard, the instructions can cause the processor 104 to receive c-peptide measurements to generate newly received c-peptide data. For example, the system 100 can include a c-peptide measurement device 112 configured to generate the newly received c-peptide data. The c-peptide measurement device 112 can be in communication with the processor 104 or in communication with a data store 103 that is in communication with the processor 104. The c-peptide measurement device 112 can be a urine sample test (e.g., an assay device).

Embodiments can relate to a method for managing a database for efficient physiological diagnosis of a specified physiological disorder. The method can involve receiving a glucose measurement possibly associated with controlled glycemic-response consumption activity. The method can involve searching a physical data store 102 by comparing a newly received glucose measurement trace to at least one classification using a similarity metric. The physical data store 102 can contain glucose measurement data and a representation for the at least one classification of the glucose measurement data. The glucose measurement data can be associated with at least one controlled glycemic-response consumption activity. The representation can be an indication that a glucose measurement trace from the glucose measurement data is associated with a specified physiological disorder or is indicative of the specified physiological disorder. The method can involve classifying the newly received glucose measurement trace with the representation based on a matched similarity metric in response to the comparing. The method can involve ascribing a clinical recommendation or assessment (e.g., diagnosis, risk score, disease progression level, estimate for a specific metabolic test outcome, disease-specific test outcome, treatment, etc.) based on the representation of the classified newly received glucose measurement trace.

The treatment recommendation can include initiating, modifying, or forgoing administration of synthetic insulin.

It is contemplated for the controlled glycemic-response consumption activity to include consumption of the energy drink in lieu of a meal.

The physical data store can contain glucose measurement data from one or more of: individuals that have low risk of the specified physiological disorder and not diagnosed with the specified physiological disorder; individuals that have high risk of the specified physiological disorder and not diagnosed with the specified physiological disorder; or individuals diagnosed with the specified physiological disorder. The newly received glucose measurement trace is from: an individual that has low risk of the specified physiological disorder and not diagnosed with the specified physiological disorder; an individual that has high risk of the specified physiological disorder and not diagnosed with the specified physiological disorder; or an individual diagnosed with the specified physiological disorder.

### EXAMPLES

The following describes exemplary development, implementations, and testing of embodiments of the system 100 and methods disclosed herein.

The examples discussed herein leverage the significant background of the UVA CDT in the analysis of continuous glucose monitor (CGM) traces and extracting actionable metrics from these traces in a non-traditional way. In particular, we use advanced machine learning (ML)-based methods for analysis of CGM traces as part of an alternative methodology for T1D risk detection. We are motivated, in part, by a recent study that showed how a simple CGM-derived metric (average time spent above 140 mg/dL) is associated with a high risk of progression to clinical T1D within the next year in islet autoantibody-positive children; the metric also separated progressors from non-progressors. It was also suggested that “ . . . . CGM should be included in the ongoing monitoring of high-risk children and could be used as potential entry criterion for prevention trials”.

Our UVA CDT experience with CGM technology and analysis of CGM data is based on more than 15 years of development of the UVA Artificial Pancreas program. This experience includes the development of innovative methods and CGM-based metrics for assessment of glycemic control and T1D risk and the use of CGM to inform closed-loop glucose control algorithms in T1D. Most recently, we have focused our efforts on using both ML and artificial intelligence (AI) (e.g., data science) techniques for analysis and classification of daily CGM profiles relevant to prediction of T1D risk in healthy people. Currently, we are employing fuzzy C-means clustering to analyze CGM home traces and distinguish between people at different level of immunological risk. The research leading to the present invention focused on using existing data to design a self-administered, CGM-based, at-home test that predicts the T1D risk, which can but does not have to use these clustering methods.

### Example 1

This example demonstrate how one-week of continuous glucose monitoring (CGM) data from a home test can be used to accurately characterize differences in glycemia in at-risk healthy individuals based on autoantibody presence. It also demonstrates effective development of a machine-learning technology for CGM-based islet autoantibody classification, which can be used to predict the risk for type 1 diabetes (T1D).

### Methods

Sixty healthy relatives of people with T1D with mean±SD age of 23.7±10.7 years, HbA1c of 5.3±0.3%, and BMI of 23.8±5.6 kg/m² with zero (N=21), one (N=18), and ≥2 (N=21) autoantibodies, were enrolled in an NIH TrialNet ancillary study. Participants wore a CGM for a week and consumed three standardized liquid mixed meals (SLMM) instead of three breakfasts. Glycemic outcomes were computed from weekly, overnight (12:00-06:00), and post-SLMM CGM traces, compared across groups, and used in four supervised machine-learning autoantibody status classifiers. Classifiers were evaluated via 10-fold cross-validation using the receiver operating characteristic area under the curve (AUC-ROC) to select the best classification model.

### Results

Among all computed glycemia metrics, only three were different across the autoantibodies groups: percent time >180 mg/dL (T180) weekly (p=0.04), overnight CGM incremental AUC (p=0.005), and T180 for 75 min post-SLMM CGM traces (p=0.004). Once overnight and post-SLMM features are incorporated in machine-learning classifiers, a linear Support Vector Machine (SVM) model achieved the best performance of classifying antibody positive vs. antibody negative participants with AUC-ROC ≥0.81.

### Conclusion

A new technology combining machine learning with a potentially self-administered one-week CGM home test can help improve T1D risk detection without the need to visit a hospital or use a medical lab.

### INTRODUCTION

The progression towards clinical type 1 diabetes (T1D) can be categorized into three stages; the first stage is characterized by the presence of ≥2 islet autoantibodies with normoglycemia, the second stage progresses to dysglycemia (i.e., at-risk), and finally the third stage is defined by the onset of symptomatic (i.e., clinical) T1D. Therefore, the presence of autoantibodies is related to the immunological risk of developing diabetes in the future, and is a key biomarker of the pathogenic processes leading to clinical diagnosis. This, and also other, biomarkers could be used to screen a much broader population besides individuals at increased genetic risk of T1D (e.g., first-degree relatives).

Early identification and screening of individuals at increased T1D risk can reduce the rates of diabetic ketoacidosis (DKA) at diagnosis, improve the quality of glycemic control, reduce other future poor health outcomes, and complications. Overall, as monitoring of high-risk individuals in natural history studies markedly reduces DKA rates at diagnosis, research participation in these studies is critical to finding means of preventing or delaying T1D and justifies the development of efficient screening methods to identify individuals at high T1D risk which appears influenced by immunological and genetic factors. Screening for genetic T1D risk can be performed as a self-administered at-home test, but this test does not directly account for the immunological risk (i.e., presence of autoantibodies). Screening individuals for genetic risk, followed by autoantibody testing may improve the predictive power of a positive antibody test but can still miss many individuals that will develop T1D in the future. Although ~90% of those who develop T1D have no family history of the disease, this genetic predisposition puts individuals with first-degree relatives at a 20-fold higher risk of developing T1D. The current approach for T1D risk detection includes testing for presence of autoantibodies with the presence of multiple autoantibodies being more predictive of future T1D than a single autoantibody.

Recently, several studies employed continuous glucose monitoring (CGM) devices not only in people with diabetes but also in obese individuals and in individuals at different stages of prediabetes. Studies have suggested that CGM devices can be used for detecting early hyperglycemia in children with multiple autoantibodies, and for predicting progression to diabetes in antibody positive (Ab+) children. CGM can be included in the ongoing monitoring of high-risk children (Ab+), where they used home-based CGM wear without any additional metabolic testing (e.g., mixed meal tolerance test (MMTT)). In addition, in type 2 diabetes (T2D), CGM is able to detect impaired glycemia in certain categories of participants, earlier than other standard biomarkers used for the diagnosis and classification of diabetes. Recently, a one week CGM test has been investigated for its ability to be used for identifying individuals at higher risk for rapid progression to Stage 3 T1D, including in those with a normal oral glucose tolerance test (OGTT). This study has identified several CGM-derived metrics of hyperglycemia associated with progression to Stage 3 disease.

Machine learning techniques have been utilized in the field of diabetes, especially in applications using CGM data to develop predictive models that could help clinicians improve screening and treatment. A logistic regression model with glycemic variability features extracted from CGM signals was used to classify individuals with and without diabetes. Several established machine-learning models for binary classification were used to classify the quality of overnight glycemic control in T1D. Prior to the presently disclosed innovation, the machine learning methodology for using CGM-based glycemic features to predict if a healthy individual is antibody positive or antibody negative (Ab+ or Ab−) as an alternative to the standard test for islet autoantibodies has not been explored.

At-home testing for disease risk could help address many of the challenges regarding whom to screen for T1D risk using antibody testing. The objective of this work is to characterize the CGM traces in healthy individuals with different number of islet autoantibodies and use CGM-based metrics to develop a (pre) screening technology to classify participants autoantibody status (Ab+vs. Ab−). The new technology uses a dedicated machine learning methodology and a, potentially self-administered, one-week CGM home test that includes up to three standardized liquid mixed meals (SLMM) challenges.

### Materials And Methods

The NIH-funded TrialNet ancillary study (ClinicalTrials.gov registration no. NCT02663661) enrolled healthy relatives of people with T1D with different numbers of islet autoantibodies, zero, one, or two or more recruited from participants with known autoantibody status in the TrialNet Pathway to Prevention study (https://www.trialnet.org/our-research/risk-screening). Major inclusion criteria included individuals 12 to 45 years old who had a brother, sister, child, or parent with T1D, or individuals 12-20 years old who had a cousin, aunt, uncle, niece, nephew, half-brother, half-sister, or grandparent with T1D. Among the major exclusion criteria were diagnosis of diabetes (i.e., T1D or T2D), a relevant medical condition (e.g., gastroparesis), or being treated with medications that might interfere with the study. All participants signed an informed consent. Participants were asked to come to the Clinical Research Unit (CRU) at the University of Virginia for a 10-hour inpatient visit (a single 10-hour clinical test consisting of a MMTT followed by insulin-induced hypoglycemia).

At the end of the hospital visit, the participants were given a blinded Dexcom G4 Platinum CGM, which they wore for the next seven days at home. They were asked to calibrate their CGMs according to the manufacturer's instructions. During this period, they consumed standardized liquid mixed meals (SLMM; Boost, Nestlé, Switzerland) over 1-5 minutes on three occasions to replace their breakfasts (6 mL/kg body weight to a maximum of 360 mL) and recorded its timing to link the start of the SLMMs with the CGM profiles. In this work, we focus solely on the CGM home study.

### CGM-Based Glycemia Metrics and Group Comparison

The CGM-based metrics and characterization of glycemia in the different autoantibodies groups was performed under three different scenarios: OVERALL (based on all 7 days), OVERNIGHT (based on all 7 days overnight periods), and PostSLMM (based only on the post-SLMM CGM traces) as described below.

### Overall

CGM data from the participants were collected and glycemic features/metrics were extracted and computed including; mean glucose (MG), percent time of glucose >180 mg/dL (T180), >160 mg/dL (T160), >140 mg/dL (T140), <70 mg/dL (T70), <54 mg/dL (T54), coefficient of variation (CV), standard deviation (SD), range, low blood glucose index (LBGI, measures the frequency and magnitude of hypoglycemia), high blood glucose index (HBGI, measures the frequency and magnitude of hyperglycemia), and the average daily risk range (ADRR, the sum of the daily peak risks for hypo- and hyperglycemia) [see, Table 1]. In more detail, ADRR is a variability metric based on “risk” values obtained from glucose levels that are mathematically transformed to give equal weight to hyperglycemic and hypoglycemic excursions. LBGI and HBGI are based on the same normalizing transformation as the ADRR, but are designed to be sensitive to hypoglycemia or hyperglycemia, respectively. These metrics were used to characterize the glycemic responses of participants in different autoantibody classes.

### Overnight

Twelve glycemic features were extracted and computed from the overnight (12:00-06:00) CGM traces. These features include; MG, T180, T160, T140, T70, T54, CV, SD, range, LBGI, HBGI, and the AUC above the baseline value at midnight (the overnight CGM incremental area under the curve (IAUC)), in order to characterize the glycemic responses in the different autoantibodies groups.

### PostSLMM

We investigated the length of 0-2 h post-SLMM to get a significant difference post-SLMM excursion between the different autoantibodies groups by using nine glycemic features: CV, T140, T160, T180, the AUC above the baseline value at t=0 (IAUC), Glucose level at t min post-SLMM (Gt), maximal glucose amplitude (Gmax), time to Gmax (Tmax), and slope of glucose 0−t min(S). Those features capture the dynamic characteristics of the post-SLMM CGM dataset for each participant in the three different autoantibodies groups.

### Statistical Procedures

All statistical analyses were performed using R Statistical Software 4.0.2 (R Foundation for Statistical Computing). The Shapiro-Wilk test was used to check if glycemic features follow a normal distribution. For normally distributed continuous variables, a one-way analysis of variance (ANOVA) was used to compare the means between autoantibodies groups. For non-normally distributed variables, a Wilcoxon signed-rank test and Kruskal-Wallis were used to determine whether there are statistically significant differences between the glycemic features in different autoantibodies groups. Bonferroni correction was used for multiple comparisons correction to reduce the chances of obtaining false-positive results. A p-value <0.05 was considered to be significant. Pearson's correlation matrix between the glycemic features was computed, to assess the collinearity between glycemic features.

### Autoantibodies Classification

The extracted glycemic features from the three different scenarios were used to define different classifier models based on the autoantibodies class. Then, these features were aggregated per participant and each feature was mean-centered and scaled before entering the classification procedure.

We merged 1 autoantibody with ≥2 autoantibodies in one class as an autoantibody positive “Ab+” class vs. the autoantibodies negative class “Ab−”. Two different options for using glycemic features in the classifiers models were investigated; either using the significant features only (i.e., glycemic features that are statistically significant differences between the autoantibodies groups) or using all the glycemic features in the three different scenarios based on the autoantibodies class.

### Classification Models

Four different classification models were used to develop an autoantibodies classifier and define the best classifier model: linear discriminant analysis (LDA), linear support vector machine (SVM), logistic regression (LR), and K-nearest neighbors (KNN).

### Classification Strategy

A 10-fold cross-validation technique was implemented. The entire data set of glycemic features from all participants is aggregated per participant and is randomly shuffled. Then, it was subdivided into 10 approximately equal-sized folds/sections. One of the 10 folds was used as a test set to evaluate classification performance, while the remaining 9-folds were used to train the classifier models. The procedure was repeated 10 times (iterations) to estimate the mean performance of the different classifier models. This procedure guarantees that data from each participant either appears either in the training or in the test set (but not both), avoiding overfitting and improving the generalizability of the results.

### Class Imbalance

Class imbalance refers to a classification predictive modeling problem when the class distribution is not equal or close to equal in the training dataset (i.e., a significantly larger proportion of Ab+ than Ab−), and is instead biased or skewed. This can result in biased predictions and misleading accuracies. We address the class imbalance (i.e., unbalanced samples) by using up-sampling of the minority class (oversampling) only in the training dataset. In our experiments, oversampling was performed within rather than before the 10-fold cross-validation technique to ensure that no participant is in both the training and test sets, and thereby avoid the overestimation of the model performance.

### Classification Performance Assessment

To assess the performances of classifier models, a confusion matrix was used to report the four possible outcomes of the comparison between the true and the predicted class, that is, true negative (TN), false negative (FN), true positive (TP), and false positive (FP). The receiver operating characteristic area-under-the-curve (AUC-ROC) was used to select the best-performing classifier models. AUC-ROC is a numerical index that depicts the trade-off between the Sensitivity (i.e., True positive rate) and (1-Specificity) (i.e., False positive rate) across a series of different cut-off points, which are given by

$$Sensitivity = \frac{T P}{( T P + F N )}$$

$$1 - Specificity = \frac{F P}{( F P + T N )}$$

The closer to 1 the AUC-ROC, the better the classifier model at distinguishing between Ab+ vs. Ab− participants.

### Results

Seventy-three participants were recruited for this study, and stratified into three groups with zero (N=25), one (N=21), and ≥2 (N=27) autoantibodies. One participant was diagnosed with diabetes, 5 failed screening, and 7 withdrew from the study (the screen failures/withdrawers were not related to the CGM study. Sixty participants completed the CGM study and were included in the analysis. Of these participants, 21, 18, and 21 had zero, one and more than one autoantibody, respectively. They had mean±SD age of 23.7±10.7 years (range 12-42 years), HbA_{1c} of 5.3±0.3%, and BMI of 23.8±5.6 (kg/m²) (Table 1). There were no statistically significant differences between the three groups with regard to these characteristics.

TABLE 1
Clinical and demographic characteristics of sixty participants
in the three different classes of islet autoantibodies (Ab) used
for analysis. Statistics presented as N, mean (SD), or (%).

| | Negative | | | | 2 or more | |
|---|---|---|---|---|---|---|
| Characteristic | (Zero Ab) | | 1 Ab | | Ab | |
| Number of subjects (N) | 21 | | 18 | | 21 | |
| Sex, % female | 66.7 | | 50.0 | | 57.1 | |
| Age (years) | 27.0 | (9.9) | 23.5 | (11.9) | 20.7 | (10.2) |
| Race, % White/ | 100 | | 100 | | 100 | |
| Caucasian | | | | | | |
| HbA_{1c} (%) | 5.3 | (0.3) | 5.3 | (0.3) | 5.3 | (0.3) |
| BMI (kg/m²) | 23.7 | (5.3) | 25.3 | (6.5) | 22.8 | (5.1) |

### Overall CGM-Based Glycemia Dynamics

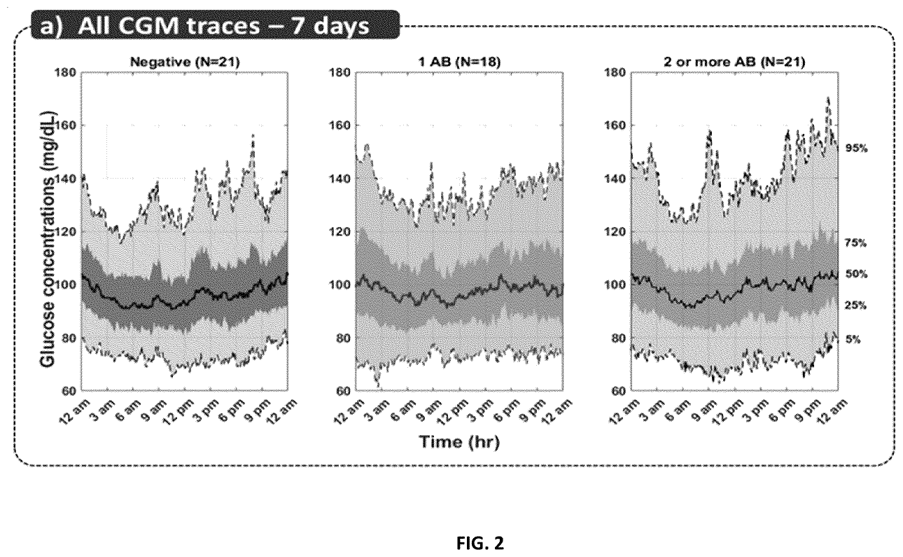

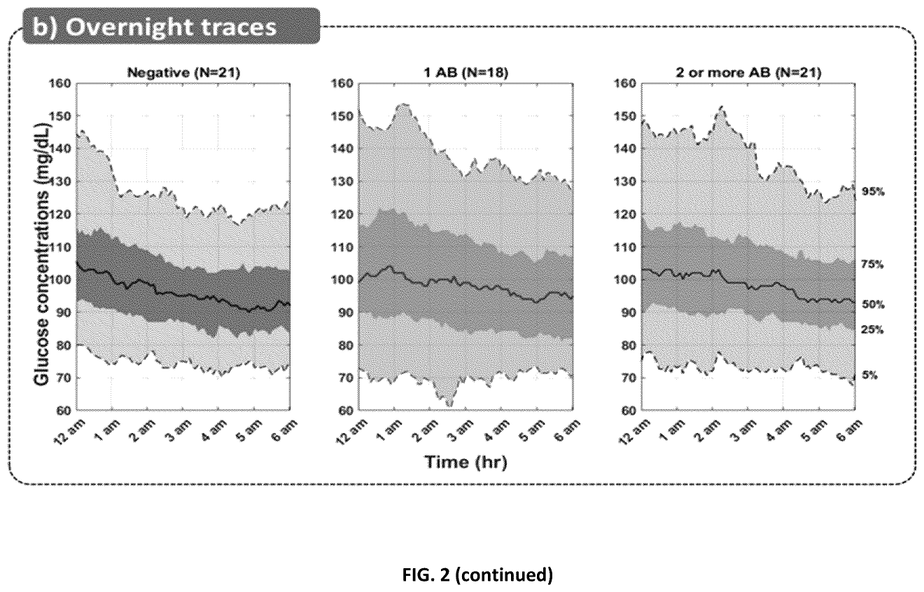

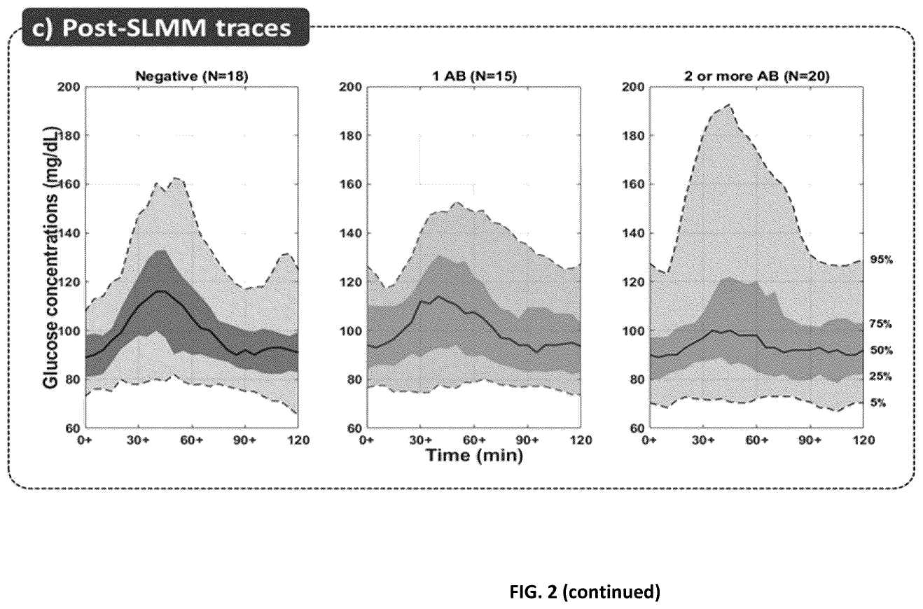

The average CGM in the three different scenarios were not significantly different between the three groups which illustrates the difficulties of using those profiles to characterize the glycemic responses of participants in different groups of autoantibodies, as the single ambulatory glucose profile (AGP) visual display in the three different scenarios are not apparently distinct (see FIG. 2), except panel c (Post-SLMM) is actually distinct-appearing for the 2 or more AB group. It appears the height of the peak, as well as the distribution of CGM traces is different from the other two groups (i.e., Negative and 1 AB). FIG. 2 represents three different panels of CGM traces aggregated to create a single ambulatory glucose profile (AGP) as a visual display in different autoantibodies (Ab) groups (i.e., Negative, 1 Ab, and 2≥Ab). Panel (a) shows CGM traces of the entire 7 days for 60 participants in the three different groups of Ab. Panel (b) shows CGM traces of the overnight periods (i.e., 12:00-06:00) for 60 participants in the three different groups of Ab (blow-up of the first 6 hours plotted in panel (a). Panel (c) shows CGM traces of the 2 h-post mixed meal tolerance test (MMTT) for 53 participants in the three different groups of Ab. The solid line in each Ab group in the three different scenarios is the median or 50% line; half of all CGM values are above and half are below this value. The 25th and 75th percentile curves represent the interquartile range or 50% of all CGM values. The dashed outer lines (the 5th to 95th percentile curves) indicate that only 5% of CGM readings were above or below these values in the three different scenarios. N, represents the number of participants in each group.

### Characterization of Glycemia of the Three Autoantibodies Groups Based on the Complete Seven-day CGM Data:

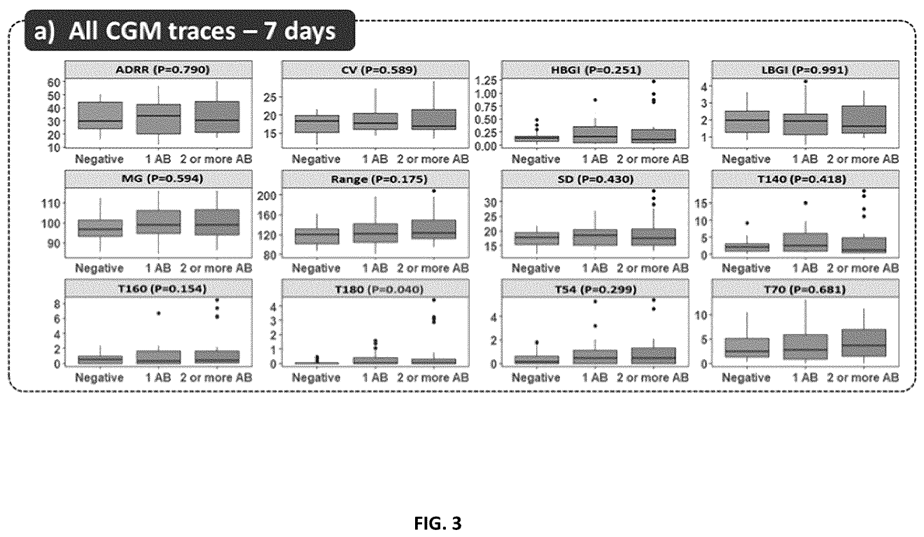

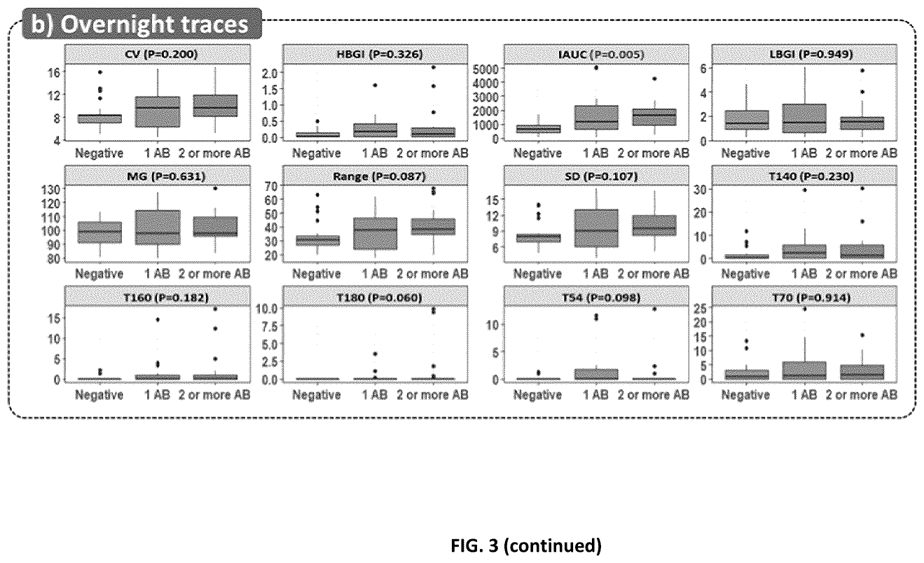

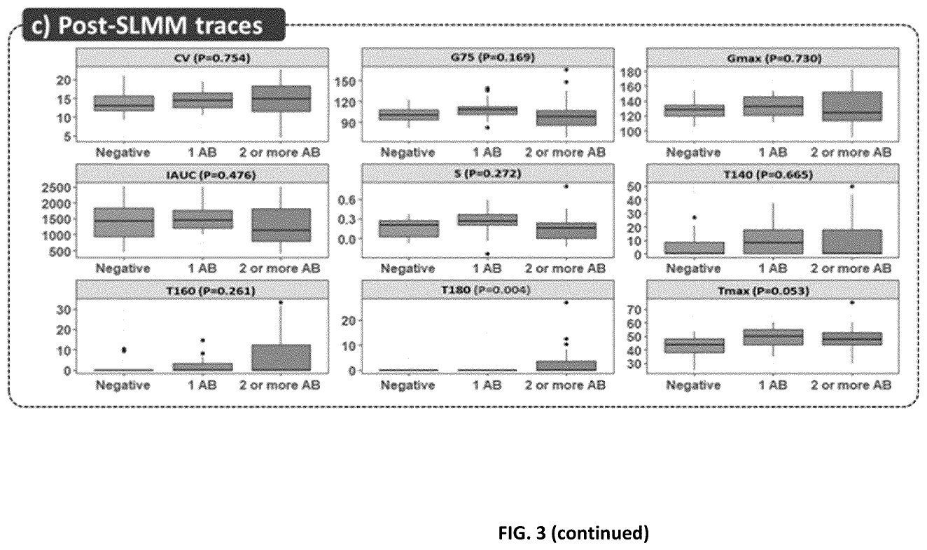

Twelve glycemic features were extracted and computed as described in Methods (OVERALL). T140, T160, T180, SD, Range, and HBGI were highly correlated (r≥0.83). There are no statistically significant differences between these twelve glycemic features in the three groups except for T180 with p=0.040 (i.e., negative vs. 1 autoantibodies with p=0.352, negative vs. ≥2 autoantibodies, with p=0.012, and 1 autoantibodies vs. ≥2 autoantibodies, with p=0.144), as shown in FIG. 3. Therefore, weekly CGM traces revealed different glycemic patterns among autoantibodies groups only through T180. FIG. 3 shows a characterization of CGM data through different glycemic features in different scenarios. Panel (a) shows boxplots for 12 different glycemic features extracted from the entire 7 days of CGM traces for 60 participants in the three different groups of autoantibodies (Ab). Panel (b) shows boxplots for 12 features extracted from overnight (i.e., 12:00-06:00) CGM traces for 60 participants in the three different groups of Ab. Panel (c) shows boxplots for 9 features extracted from 75-min post-MMTT CGM traces for 53 participants in the three different groups of Ab. Abbreviations: ADRR, average daily risk range; CV, coefficient of variation; HBGI, high blood glucose index; LBGI, low blood glucose index; MG, mean glucose; SD, standard deviation; T140, percent time >140 mg/dL; T160, percent time >160 mg/dL; T180, percent time >180 mg/dL; T54, percent time <54 mg/dL; T70, percent time <70 mg/dL; IAUC, incremental area under the curve (mg/min/dL); G75, Glucose level at 75 min post-SLMM; Gmax, maximal glucose amplitude; Tmax, corresponding time to Gmax (Time [min]); S, slope of glucose 0-75 min (mg/dL)/min. A significance level of 5% (p-value <0.05) was considered to be significant to distinguish between the different groups of Ab.

### Characterization of Glycemia of the Three Autoantibodies Groups Based on Seven-Day Overnight CGM Data

Overnight CGM traces with a 6-hr duration from 12:00 am to 6:00 am were extracted from 60 participants. A total of 406 overnight CGM traces were extracted, and then a set of 12 glycemic features mentioned above were extracted and computed. Fifty participants had seven, 6 participants had six, and 4 participants had five days of overnight traces. IAUC was the only statistically significant difference between the glycemic features in the three different autoantibodies groups, with higher IAUC for those with ≥2 autoantibodies (i.e., Negative vs. 1 autoantibody with p=0.012, Negative vs. ≥2 autoantibodies, with p=0.005, and 1 autoantibodies vs. ≥2 autoantibodies, with p=0.012), as shown in FIG. 3. In addition, T180 and Range with p=0.060, p=0.087 respectively, almost reached significance, as shown in FIG. 3. Several metrics appear highly correlated. For example, the correlation between HBGI and T140, T160, and T180 was r≥0.89, and the correlation between Range and SD, and CV was r≥0.93. Notably, the correlation between IAUC and all other features was weak except with SD (r=0.48).

### Characterization of Glycemia of the Three Autoantibodies Groups Based on Post-SLMM Data

Post-SLMM CGM traces were extracted from 53 participants. We excluded seven participants from the analysis (3 participants from Negative group, 3 participants from 1 autoantibodies group, and 1 participant from ≥2 autoantibodies group): six of them had breakfast after SLMM, and one had breakfast 30-min before SLMM. CGM traces after the SLMM were first processed, and the suitable length to get a different post-SLMM excursion (i.e., statistically significant differences) between participants was t=75 minutes. Post-SLMM CGM traces (n=142) for 75 minutes (i.e., 47 CGM traces zero autoantibodies, 40 traces 1 autoantibodies, and 55 traces ≥2 Ab) were extracted from 53 participants, where 75.6% of those participants completed all 3 SLMM loads, 16.9% only did two sessions, and 7.5% only completed one session. Then, a set of nine glycemic features mentioned in Methods (PostSLMM) were computed. The only statistically significant difference between the glycemic features in the three different autoantibodies groups was T180 with p=0.004 (i.e., Negative vs. 1 autoantibody with p=1.000, Negative vs. ≥2 autoantibodies, with p=0.012, and 1 autoantibodies vs. ≥2 autoantibodies, with p=0.018), as shown in FIG. 3. Besides that, Tmax with p=0.053 almost reached significance, with higher Tmax for those with 1 autoantibody and ≥2 autoantibodies, as shown in FIG. 3. T140, T160, IAUC, and Gmax were highly correlated features (r≥0.71), while the correlation between Tmax and all other features was very weak, except with the slope S and G75 (r=0.53 and r=0.45, respectively).

Characterization of Glycemia of the Ab+ vs. Ab− Participants

### OVERALL

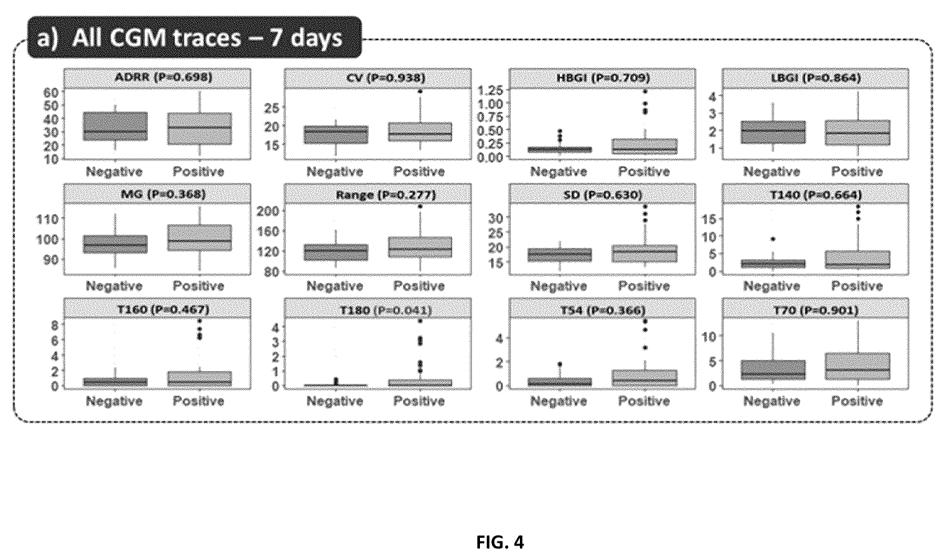

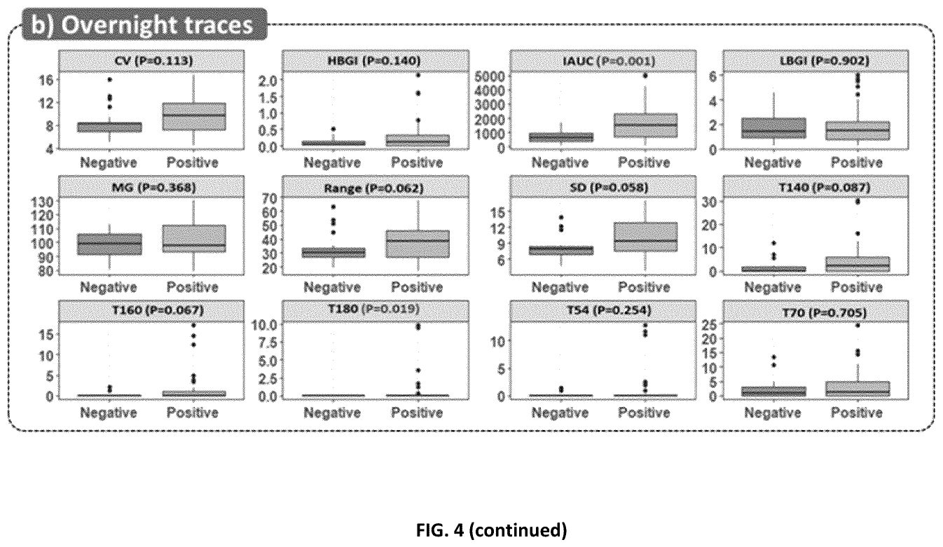

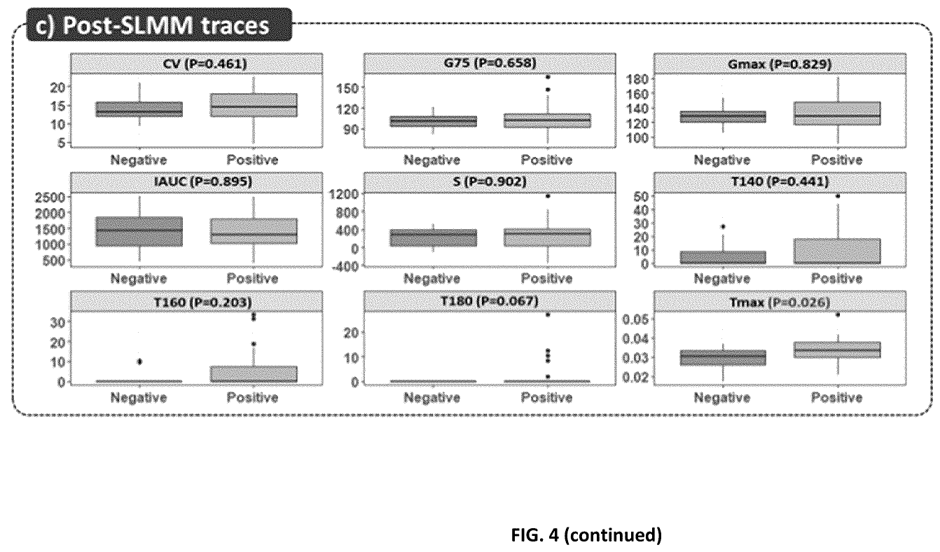

60 observations and 12 glycemic features are contained in the entire 7 days of CGM traces dataset, including 65% of all participants in the Ab+ class and the remaining 35% in the Ab− class (39 Ab+ vs. 21 Ab−). T180 of the 12 glycemic features was the only statistically significant difference between both classes with p=0.041 (see FIG. 4). FIG. 4 shows characterization of CGM data through different glycemic features in different scenarios. Panel (a) shows boxplots for 12 different glycemic features extracted from the entire 7 days of CGM traces for 60 participants in two different groups of autoantibodies (Ab+/Ab−). Panel (b) shows boxplot for 12 features extracted from overnight (i.e., 12:00-06:00) CGM traces for 60 participants in two different groups of Ab. Panel (c) shows boxplots for 9 features extracted from 75-min post-SLMM CGM traces for 53 participants in two different groups of Ab. A significance level of 5% (p-value <0.05) was considered to be significant to distinguish between the different groups of Ab.

### OVERNIGHT

60 observations and 12 glycemic features are contained in the overnight CGM traces dataset, including the same portion of participants in both autoantibodies classes as in OVERALL. The overnight CGM IAUC and T180 were statistically significant differences between Ab+ vs. Ab− with p=0.001 and p=0.019, respectively (see FIG. 4).

### PostSLMM

53 observations and 9 glycemic features are contained in the post-SLMM CGM traces dataset, including 66% of participants in the Ab+ class and the remaining 34% in the Ab− class (35 Ab+ vs. 18 Ab−). Tmax was the only statistically significant difference between both classes of autoantibodies with p=0.026 (see FIG. 4).

Defining Classifier Models Based on the Ab+ vs. Ab− Groups:

As the datasets in the three scenarios above were “imbalanced” according to the autoantibodies class distribution, we followed the balancing procedure described in Methods before applying any of the classifier models. The four binary classifier models with a 10-fold cross-validation technique and oversampling were implemented with the only significant features, and then using all the glycemic features from the three different scenarios, to classify participants in terms of presence (Ab+) or absence (Ab−) of autoantibodies.

TABLE 2
Comparison of classification performance of four models with oversampling technique
in terms of AUC-ROC based on different groups of autoantibodies (i.e., Ab+
vs. Ab−) in different scenarios (i.e., using glycemic features extracted
from the entire 7 days of CGM traces vs. features extracted from overnight
(i.e., 12:00-06:00) CGM traces vs. features extracted from 75-min post-SLMM
CGM traces vs. mixing overnight features & SLMM features), when we defined
the four models by using only the significant features for each scenario.

| | | | | AUC-ROC |
|---|---|---|---|---|
| | AUC-ROC | AUC-ROC | AUC-ROC | Overnight& |
| | Overall CGM | Overnight | SLMM | SLMM |
| Classification | (1 feature; | (2 features; | (1 feature; | (3 features; |
| models | T180) | IAUC, T180) | Tmax) | IAUC, T180, Tmax) |
| Linear | 0.627 | 0.754 | 0.789 | 0.778 |
| Discriminant | | | | |
| Analysis | | | | |
| (LDA) | | | | |
| Support | 0.671 | 0.758 | 0.777 | 0.811 |
| Vector | | | | |
| Machine | | | | |
| (SVM) + | | | | |
| Linear Kernel | | | | |
| Logistic | 0.657 | 0.794 | 0.789 | 0.786 |
| regression | | | | |
| (LR) | | | | |
| K-nearest | 0.627 | 0.661 | 0.728 | 0.777 |
| Neighbors | | | | |
| (KNN) | | | | |

### OVERALL

The linear SVM classifier model outperforms the other classifier models with a mean AUC-ROC of 0.67, when using T180 as a significant feature, to classify those participants in different autoantibodies classes, as shown in the first column of Table 2. Using the 12 extracted features in the four binary classifier models did not improve the classification accuracy, as shown in the first column of Table 3, where the LR classifier model outperforms the other classifier models with a mean AUC-ROC of 0.69.

TABLE 3
Comparison of classification performance of four models with oversampling
technique in terms of AUC-ROC based on different groups of autoantibodies
(i.e., Ab+ vs. Ab−) in different scenarios, when we defined
the four models by using all the features for each scenario.

| | AUC-ROC | | | AUC-ROC |
|---|---|---|---|---|
| | Overall | AUC-ROC | AUC-ROC | Overnight& |
| | CGM | Overnight | SLMM | SLMM |
| Classification models | (12 features) | (12 features) | (9 features) | (21 features) |
| Linear Discriminant Analysis | 0.679 | 0.679 | 0.804 | 0.693 |
| (LDA) | | | | |
| Support Vector Machine | 0.672 | 0.812 | 0.825 | 0.776 |
| (SVM) + Linear Kernel | | | | |
| Logistic regression (LR) | 0.692 | 0.765 | 0.778 | 0.715 |
| K-nearest Neighbors (KNN) | 0.639 | 0.621 | 0.776 | 0.760 |

### OVERNIGHT

Using IAUC and T180 as significant features leads to a noticeable improvement as shown in the second column of Table 2, where the LR classifier model outperforms the other classifier models with a mean AUC-ROC of 0.79. While, using the extracted 12 features, leads to a notable improvement in classification accuracy, where a linear SVM classifier model outperforms the other classifier models with a mean AUC-ROC of 0.81, as shown in the second column of Table 3.

### PostSLMM

Using Tmax only as a significant feature lead also to a noticeable improvement, as shown in the third column of Table 2. LR and LDA classifier models outperform the other classifier models with a mean AUC-ROC of 0.79. More improvement was achieved when using the 9 features, where a linear SVM classifier model outperforms the other classifier models with a mean AUC-ROC of 0.83, as shown in the third column of Table 3.

In addition, using the significant features from OVERNIGHT and PostSLMM together (i.e., T180, IAUC, and Tmax), improved the classification accuracy, and a linear SVM classifier model outperforms the other classifier models with a mean AUC-ROC of 0.81, as shown in the fourth column of Table 2. However, mixing all the extracted features from both scenarios did not improve the accuracy of classification, as shown in the fourth column of Table 3.

### Discussion

In this work, we used data from a recent NIH-funded TrialNet ancillary study using relatives of people with T1D of 12-42 years of age to characterize the extent to which features derived from a one-week CGM home test can stratify individuals with different number of T1D-specific autoantibodies. While standard metrics, such as MG, SD, and CV were unable to stratify the different autoantibodies groups in the overall seven days or overnight CGM traces, T180 based on the overall seven days CGM traces distinguishes between the three autoantibodies groups, which was also the case for the CGM IAUC based on the overnight CGM traces, where IAUC was lower in the Ab− group vs. Ab+. Besides, the post-SLMM periods T180 was a statistically significant difference between the three autoantibodies groups, and Tmax approached significance. Therefore, the highest glucose excursions (T180) appear as a metric that differentiates between the three autoantibodies groups, likely driven by different meal responses. This is in line with what was observed previously for children with median age 11.5 years, with the caveat that in our study, T140 was not as predictive as T180. On the other hand, the ability of overnight IAUC to distinguish between the different groups suggests the ability of the participants with a lower number of autoantibodies to reach faster their baseline glucose values. The data collected during the home CGM study and the glycemia metrics/features derived from it allowed the use of machine-learning methodology to develop an autoantibodies status classifier. Notably, features based on the complete seven-day CGM traces, were unable to classify with sufficient accuracy the Ab+ vs. Ab− participants, but the overnight and post-SLMM CGM traces were able to better capture the differences between the groups. Glycemic features extracted from the overnight and post-SLMM CGM traces were able to distinguish the Ab+ vs. Ab− participants, and predict the autoantibodies status with only a small number of significant features like T180 and IAUC from the overnight traces, and Tmax from the post-SLMM traces. The disclosed methodology of the autoantibody classifier, which combines the CGM home test data with a linear SVM-based classifier, was able to predict with high accuracy (i.e., AUC-ROC≥0.81) the participant's presence or absence of autoantibodies. Overall, these results support the notion that adding the SLMM intervention to the home CGM test improves our ability to use the test to distinguish Ab+ vs. Ab− participants with a small number of features with different but complementary physiological meaning. We also note that the disclosed technology allows addressing not only the question of classifying Ab+ vs. Ab−, but also exploring the option for classifying low-risk (zero and one autoantibody) vs. high-risk (two and more autoantibodies; Stage 1 and 2).

As mentioned herein, a recent study in individuals in T1D probands has identified several CGM-derived metrics of hyperglycemia significantly associated with rapid progression to Stage 3 disease, including in those with normal OGTT results. These metrics are based on selected percent time (5% or 8%) with glucose above different glucose level thresholds (e.g., glucose over 120, 140, and 160 mg/dL). Even though our technology is not tailored to stratify progressors to Stage 3 from non-progressors, it identifies new metrics derived from the overnight and post-SLMM CGM periods that can be explored to estimate the imminent risk for progression to Stage 3 T1D.

The CGM home test can be self-administered after a carefully designed interactive online teaching session and would not require a visit to a health care facility or use of a medical lab. Therefore, it could be used as an alternative or in addition to current home screening methods like the GTT@ home (https://www.digostics.com) and self-collected capillary blood autoantibodies test currently employed by TrialNet. It can provide additional information on the level of dysglycemia that cannot be obtained by a single-finger stick for autoantibody presence or a genetic test. It is contemplated for the test to complement other T1D risk biomarkers (including genetic), to estimate better the autoantibodies status, and the overall risk of developing T1D, and/or separate progressors from non-progressors in autoantibody-positive individuals. Ultimately, this could provide insight towards onset of therapy, potentially avoiding cases of DKA and highlighting individuals who could benefit from future immune-modulatory interventions such as teplizumab.

### Conclusion

In conclusion, in the early stages of progression to T1D, a CGM-based test can reveal increasing levels of dysglycemia, which may be too subtle in the beginning to cause any visible symptoms, but their progression over time could lead to early diagnosis and avoidance of DKA and hospital admissions. In the very early stages of the disease, standard glycemia metrics derived from a one-week CGM home test were able to differentiate between individuals at different autoantibodies status through different scenarios. Using machine learning further allowed to develop a method to distinguish CGM patterns between individuals without vs. with T1D antibodies, based on assessment performed at home. If applied broadly, this approach could help improve T1D risk detection, potentially alerting individuals for early diagnosis or prevention.

### Example 2

While embodiments disclosed herein can utilize machine learning to classify CGM data and predict T1D risk without use of clustering techniques discussed in EXAMPLE 1, EXAMPLE 2 demonstrates that clustering techniques can be used.

### Research

We use CGM traces generated by our dedicated, self-administered CGM one-week at-home test with consumption of three liquid caloric drinks (boosts) as a mixed meal tolerance test (MMTT), subsequently referred to as the CGM/MMTT. We use existing CGM/MMTT data to develop a new technology for screening that can identify reliably both the immunological and genetic risk to develop T1D. We use two different ML/AI clustering techniques for analysis of the CGM traces that are either available, and are the training set, or will become available during the course of the project. These techniques can allow for use of additional training and testing data for validation of the methodology.

### Rationale

Developing simple methods for screening individuals for their immunological and genetic risks to develop T1D could provide significant benefits for both research and clinical practice. This research takes advantage of two critical circumstances unique to our group at UVA. First, we are likely the only Center that has data that combine: a home CGM test, knowledge of individuals immunological and genetic risks, and additional data, less relevant to the current application, like hospital OGTT data, and inpatient 8-hour frequent sampling data for analysis of the individual's hormonal dose-response interactions responsible for maintaining their glucose homeostasis. Second, we have more than 15 years of experience in development of innovative mathematical and engineering methods and CGM-based metrics for assessment of glycemic control and risk. Recently, we developed two advanced data science methods for analyzing the CGM traces generated by our CGM/MMTT at-home test. The first is universal, applicable to the entire set of daily CGM traces and used to classify people at risk. The second is test-specific and takes full advantage of the fact that the subjects had consumed three identical caloric drinks during the one-week CGM test. The ability of these two methods to evaluate our CGM/MMTT test data and distinguish between individuals at different immunological risk provides the rationale to further develop and validate these approaches as a combination of a dedicated one-week CGM test plus advanced methodology for analysis of its results.

### Research Design And Methods

This application includes is to design of machine-learning/artificial intelligence (ML/AI) data science methodology for analysis of a dedicated CGM at-home test to distinguish between individuals at different immunological and genetic risk for T1D.

### Aim 1

We use an in silico aim to design a new methodology for analysis of data generated by our self-administered CGM/MMTT at-home test to distinguish between people at different immunological and genetic risk to develop T1D. This unique test was originally designed to detect physiological differences in the cross-talk between glucose, insulin and glucagon in people at different immunological risk to develop T1D. During the at-home CGM/MMTT test all subjects wore a CGM for one week, consumed three mixed meal nutrition drinks (Boost) instead of three breakfasts, and had a urine sample collection (for C-peptide and creatinine) 2 hours after one of the nutrition drinks. Two sub-aims are used corresponding to Milestones 1 and 2.

### Aim 1.1

In existing data with CGM and autoantibodies to develop an algorithm for analysis of the CGM/MMTT test that can distinguish between people at different immunological and genetic risk to develop T1D.

### Aim 1.2

Assess the performance of the new algorithm with CGM/MMTT by follow-up data analysis on the NIH DP3 subjects (Farhy, PI) through the TrialNet Coordinating Center. Fine-tune and simplify the CGM/MMTT-based risk score algorithms.

Aim 1—Design of machine-learning methodology for analysis of a dedicated CGM home test to distinguish between individuals at different immunological and genetic risk to develop T1D.

### CGM/MMTT Home Test Protocol And Data Sources

Description of the home CGM/MMTT test. The data originate from other completed or ongoing studies. The design of the protocol is essentially identical in all data sources. In particular, all subjects are asked to wear a CGM for 7 days according to the manufacturer recommendations and are instructed to return study supplies after the end of the study. During the 7-day period of CGM wearing, subjects are asked to have three identical mixed meal caloric drinks (Boost; provided to subjects) at 8 am on Day 3, 5, and 7 instead of three breakfasts. Two hours after the second Boost, subjects are asked to take a urine sample in a team provided container to be mailed back to the study team for evaluating the individual's C-peptide responses to the Boost via measuring the C-peptide/creatinine ratio in the urine sample. All study participants use the most current version of DexCom CGM. Subjects are also asked to keep track of the time when they have a meal, snack, or caloric beverage during the one-week period of wearing the CGM. The data collection protocol is schematized in the figure on the right.

### Data Sources

### The CGM Home Protocol Above is Key Component of Two Studies:

Completed: NIH funded DP3 TrialNet auxiliary study (Farhy PI, Brown, Co-PI). The clinical work of this study is now completed and we are in a data analysis phase. Under this project we performed the CGM/MMTT outlined above in 61 healthy relatives to T1D stratified in three different groups: Group 1 (N=22) has zero antibodies, Group 2 (N=18) has 1 autoantibody, and Group 3 (N=21) has 2 or more autoantibodies. All data is currently available and calculating the T1D genetic risk score for Groups 2 and 3 is underway. This can be used to train the machine learning methods.

### Methodology for Analysis of CGM/MMTT Home Test Data

One model used for the analysis of the CGM/MMTT data is based on fuzzy C-means clustering and focuses on the CGM traces around each of the three Boosts. Therefore, it is test-specific and requires the subject to have three liquid mixed meals (Boosts) for the duration of the home CGM test. It is important to note that the data collecting method for the fuzzy C-means approach is more involved than data collecting method which relies on the entire CGM trace and does not require a home MMTT. The performance of the different methods can be contrasted in order to determine the level of complexity required from the CGM home test.

Method 1: Fuzzy C-Means clustering of CGM traces around each of the MMTTs. In a preliminary effort we explored whether the outcomes of the CGM/MMTT can distinguish between subjects at different levels of immunological risk to develop T1D as defined by the number of their positive islet autoantibodies (Ab+). We applied clustering techniques to detect hidden information in CGM-detected glucose behavior associated with a Boost in the Boost periods (BP) defined as a fixed time period of 3-hrs duration around each MMTT (1-hr pre- and 2-hrs post MMTT). Fuzzy C-Means clustering (FCM) algorithm was applied to all CGM traces of 3-hrs duration around each MMTT by utilizing the Euclidean distance to measure the dissimilarity between the j^{th} CGM time series and i^{th} cluster prototype. In more detail, FCM partitions CGM data into c>1 clusters by minimizing the objective function

$${J}_{m} = {min}_{( U , V )} \{ {J}_{m} ( U , V ; X ) {\sum }_{i = 1}^{c} {\sum }_{j = 1}^{n} {u}_{ij}^{m} {d}_{ij}^{2} ( {x}_{j} , {v}_{i} ) \} ,$$

where, X={x₁, x₂, . . . , xₙ} denotes data (i.e., 3-h BPs extracted from CGM time series in this case) of size n, V=(v₁, v₂, . . . , v_{c}) is a vector of unknown cluster prototypes vᵢ∈ [symbol] , d is a distance function (Euclidean distance function between j^{th} CGM time series and i^{th} cluster prototype, m∈[1, ∞) is the fuzziness parameter. Memberships are stored in the partition matrix U=[u_{ij], i=}1, 2, . . . , c, j=1, 2, . . . , n where

$${u}_{ij} ∈ [ 0 , 1 ] , {\sum }_{i = 1}^{c} {u}_{ij} = 1 , ∀ j and 0 < {\sum }_{j = 1}^{n} {u}_{ij} < n ∀ i .$$

In order to assess the average compactness and separation of fuzzy partitions and reach the optimal number of clusters (c), the Compose Within and Between scattering (CWB) validity index was used.

Variables (features) used to prove that clusters really exist were: incremental area under the curve, glucose at different time (e.g., G90 and G75 minutes after Boost), max glucose after Boost (Gmax), corresponding time to the peak after the Boost (Tmax), the slope at different time (e.g., S90 and S75 minutes after Boost), average glucose concentrations, and the time spent in different glycemic ranges (>140 mg/dL, >160 mg/dL, >180 mg/dL). Chi-squared test was used to assess the association between CGM c-clusters and autoantibody classes.

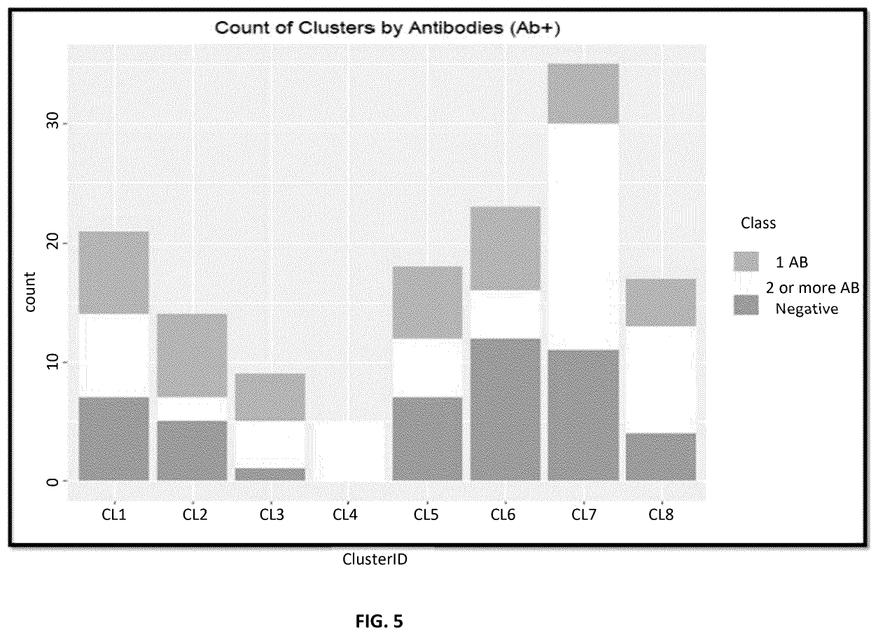

Pilot results: Eight clusters were identified by FCM and CWB validity index. A statistically significant relationship between the 8 clusters originating from the 40 (1 AB), 55 (2 or more AB), and 47 (Negative) autoantibodies, with a p-value=0.0173 was observed as plotted in the figure on the right. FIG. 5 shows the distribution of different BPs in 8 clusters based on three different categories of AB. Cluster 4 represents BPs with 2 or more AB and cluster 2 represents the majority of BPs with 1AB, while cluster 6 represents the majority of Negative AB. Therefore, this clustering technique based on the CGM/MMTT traces appears well suited for assessing the immunological risk for developing T1D.

### Developing of CGM/MMTT-Based Risk Scores

We start the analysis and the development of algorithms using existing data from the 60 healthy relatives to T1D with CGM traces and autoantibodies from the TN DP3 and genetic data for the same subjects currently underway. This data is used as initial training set to develop a classifier that uses only the three CGM traces around each Boost (from 1 hour before to 4 hours after) available for each participant to generate his/her T1D risk scores. All three CGM traces are used simultaneously and the risk score can be generated using a distance-based cluster membership in the multi-dimensional cluster space discussed above (pilot results). The algorithm is trained to assess the immunological risk for T1D.

### Additional Fine-Tuning

As we developed a CGM/MMTT-based risk scores algorithms, we will evaluate whether C-peptide measurements, which are currently part of the CGM/MMTT, are actually needed and justified. Currently, C-peptide measurements collected in the home study have not been used. We plan to re-apply both methods above by including the C-peptide data and assessing whether they contribute significantly to the algorithms predictive power. If this is not the case, the technology can be simplified and the collection of the urine sample at home can be omitted.

(ii) Additional information regarding analyses performed for EXAMPLE 2 are outlined below.

### Method 1: Clustering and Classification of CGM Traces Around Each of the MMTTs Fuzzy C-Means Clustering

In a preliminary effort we explored whether the outcomes of the CGM/MMTT can distinguish between subjects at different levels of immunological risk to develop T1D as defined by the number of their positive islet autoantibodies (Ab+). We applied clustering techniques to detect hidden information in CGM-detected glucose behavior associated with a Boost in the Boost periods (BP) defined as a fixed time period of 3-hrs duration around each MMTT (1-hr pre- and 2-hrs post MMTT). Fuzzy C-Means clustering (FCM) algorithm was applied to all CGM traces of 3-hrs duration around each MMTT by utilizing the Euclidean distance to measure the dissimilarity between the j^{th} CGM time series and i^{th} cluster prototype. In more detail, FCM partitions CGM data into c>1 clusters by minimizing the objective function

$${J}_{m} = \{ {J}_{m} = {min}_{( U , V )} \{ U , V ; X ) {\sum }_{i - 1}^{c} {\sum }_{j = 1}^{n} {u}_{ij}^{m} {d}_{ij}^{2} ( {x}_{j} , {v}_{i} ) \} \} ,$$

where, X={x₁, x₂, . . . , xₙ} denotes data (i.e., 3-h BPs extracted from CGM time series in this case) of size n, V=(v₁, v₂, . . . , v_{c}) is a vector of unknown cluster prototypes vᵢ∈ [symbol] , d is a distance function (Euclidean distance function between j^{th} CGM time series and i^{th} cluster prototype, m∈[1, ∞) is the fuzziness parameter. Memberships are stored in the partition matrix U=[uᵢⱼ], i=1,2, . . . , c, j=1, 2, . . . , n where

$${u}_{ij} ∈ [ 0 , 1 ] , {\sum }_{i = 1}^{c} {u}_{ij} = 1 , ∀ j and 0 < {\sum }_{j = 1}^{n} {u}_{ij} < n ∀ i .$$

In order to assess the average compactness and separation of fuzzy partitions and reach the optimal number of clusters (c), the Compose Within and Between scattering (CWB) validity index was used.

Variables (features) used to prove that clusters really exist were: incremental area under the curve, glucose at different time (e.g., G90 and G75 minutes after Boost), max glucose after Boost (Gmax), corresponding time to the peak after the Boost (Tmax), the slope at different time (e.g., S90 and S75 minutes after Boost), average glucose concentrations, and the time spent in different glycemic ranges (>140 mg/dL, >160 mg/dL, >180 mg/dL). Chi-squared test was used to assess the association between CGM c-clusters and autoantibody classes.

### Results

Eight clusters were identified by FCM and CWB validity index. A statistically significant relationship between the 8 clusters originating from the 40 (1 AB), 55 (2 or more AB), and 47 (Negative) autoantibodies, with a p-value=0.0173 was observed as plotted in the figure on the right. Again, FIG. 5 shows the distribution of different BPs in 8 clusters based on three different categories of AB. Cluster 4 represents BPs with 2 or more AB and cluster 2 represents the majority of BPs with 1AB, while cluster 6 represents the majority of Negative AB. Therefore, this clustering technique based on the CGM/MMTT traces appears well suited for assessing the immunological risk for developing T1D.

### Classification Model Identification for Prediction of Immunological Risk

In order to develop a classifier that can be used for prediction of immunological risk, we used the CGM traces around each of MMTTs to extract different profile features. Then, different classification models (e.g., logistic regression, support vector machines, and naive Bayes) were tested for their ability to use these features to power a classifier for immunological risk prediction. The features were either standard glycemia features (CV, T140, T160, T180, iAUC, G90/75, Gmax, Tmax, Slope) or PCA, derived from post-boost periods.

K-fold cross-validation technique was used with different classification models like neural networks and support vector machines. Cross-validation is a resampling technique to evaluate predictive models by partitioning the original dataset into a training set to train the classifier model, and a test set to evaluate it. 10-fold cross-validation was used with the nine features that extracted from CGM traces after 75 minutes of the Boost.

### Results

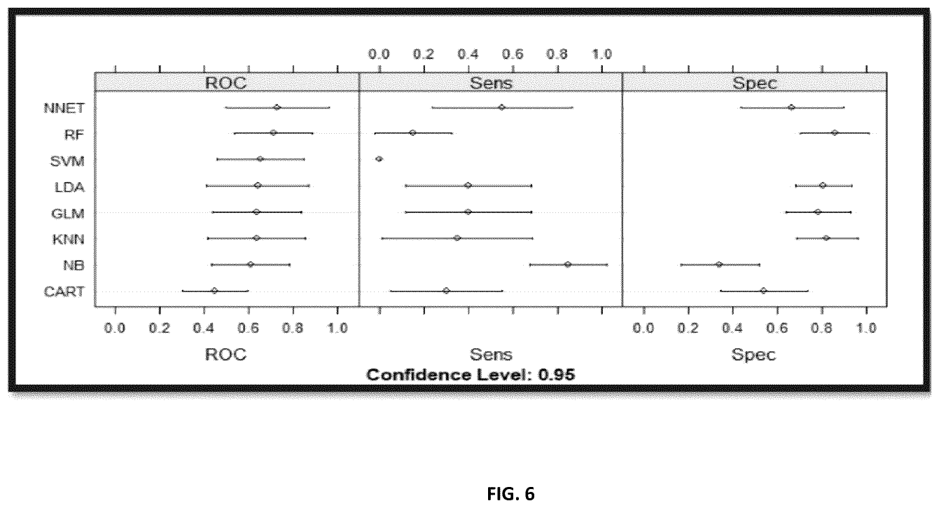

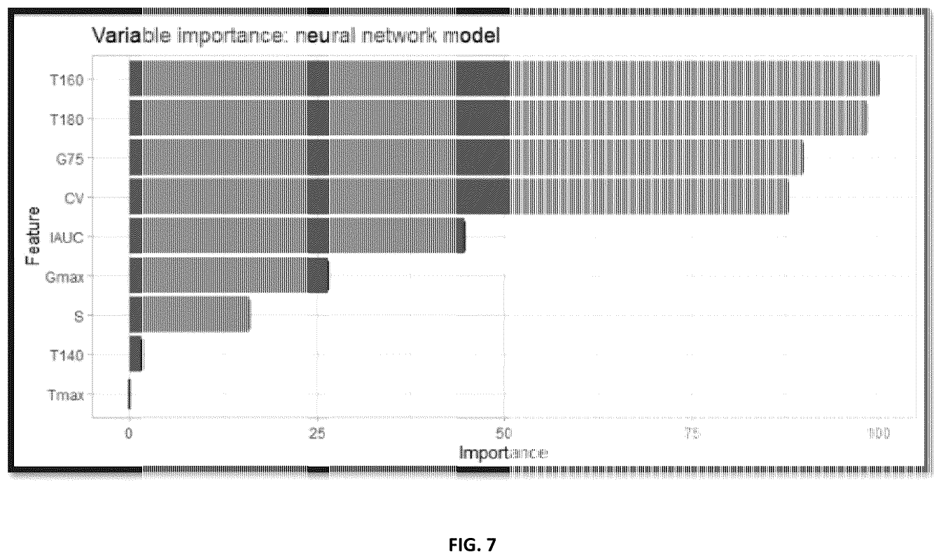

FIG. 6 summarizes the accuracy of different models through receiver-operating characteristic (ROC) curves, sensitivity, and specificity as measures to test a classifier's performance. FIG. 7 shows variable importance. As can be seen, the binary classification (autoantibody positive vs. negative) combined with neural network model as the best model (i.e., NNET), using standard glycemic features has an AUC-ROC>0.7 in detecting whether a subject have or does not have insulin autoantibodies (i.e., AUC>0.7 is considered acceptable to diagnose subjects with and without insulin autoantibodies). The result clarifies the most important features that could be used to distinguish between subjects as shown in the below figure, as well, provides a proof of concept that a dedicated CGM/MMTT in-home test can be used for screening of T1D if teamed with appropriate analytical data analysis methodology.

### Overall Comprehensive Results

In the course of this project, we have developed (among other things) a technology related to the use of CGM. We have developed a novel method for classification of daily CGM profiles that generates a limited number of classes distinguishable in their shape and clinical characteristics. The methodology can be used to enhance the performance of decision support or closed-loop systems in type 1 diabetes. We developed two advanced data science methods for analyzing CGM traces generated by our dedicated CGM/MMTT at-home test. The first is test-specific and takes full advantage of the fact that the subjects had consumed three identical caloric drinks during the one-week CGM test. The second is universal, applicable to the entire set of daily CGM traces and used to classify people at risk. The ability of these two methods to evaluate our CGM/MMTT test data and distinguish between individuals at different immunological risk has been demonstrated to a various degree thereby justifying the need to further develop and validate these approaches as a combination of a dedicated one-week CGM test plus advanced methodology for analysis of its results.

Results of the examples demonstrate:
1. CGM data can serve as a basis for different technological developments related to diabetes, potentially including prediction of next day glycemic variability in people diagnosed with the disease and in-home screening for the risk of T1D in healthy individuals
2. Dedicated CGM in-home testing together with advanced data analysis can be used to develop a screening technology for assessment of the immunological risk for T1D that is self-administered and does not require a visit to a hospital or to a lab.
3. Use of machine learning and CGM technology for prediction of next-day glycemic variability is feasible.

### Example 3

### Research

As noted herein, early screening and diagnosis of diabetes can reduce the rates of complications post-diagnosis, improve the quality of glycemic control, potentially slow the progression, reduce future poor health outcomes and long-term diabetes complications. Predicting the diabetes risk and early diagnosis of diabetes however poses a significant challenge and many efforts have been devoted to develop simple tests, including metabolic and genetic tests. Of significant clinical importance is these tests to be cheap, easy to administer, and to present minimal burden to the individual. Ideally, they should be self-administered without the need for a visit to a clinical facility or use of a clinical lab. They should also estimate not only the life-long risk for an individual to develop diabetes, but also the state of progression and the short-term risk for imminent diagnosis. For example, screening for genetic diabetes risk (both type 1 and type 2) can be performed at-home, but the test cannot account for the level of progression towards development of full-blown diabetes. Testing for presence of islet autoantibodies, can also be performed by capillary blood collection and home, but the test cannot assess the level of glucose intolerance or to estimate the imminent risk for type 1 diabetes.

Embodiments disclosed herein relate to technology for estimating the immunological risk for type 1 diabetes which uses a potentially self-administered one-week Continuous Glucose Monitoring (CGM) home test combined with tree standardized liquid mixed meals (SLMM) taken instead of three breakfasts, and machine-learning (ML)-based analysis of the collected data (CGM/SLMM/ML). The present disclosure demonstrates that the CGM/SLMM/ML technology captures subtle differences in glycemia in the very early stages of the disease and can relate these differences to the individual's immunological risk for type 1 diabetes. It is contemplated for the technology to effectively assess the imminent diabetes risk or the risk for type 2 diabetes. Example 3 includes three milestones:
Milestone 1. CGM-based Oral Glucose Tolerance Test (OGTT) replacement for diagnosis of diabetes and other metabolic disorders requiring assessment of the level of glucose intolerance.
Milestone 2. Diagnosis of diabetes with stratification between the type 1 and type 2 diabetes.
Milestone 3. Assessing the risk and level of progression (time course) of an individual towards overt type 1 or type 2 diabetes.

It is contemplated for the proposed CGM/SLMM/ML tests to use one and the same data collection protocol at home but differ in the ML methodology that will be developed for each specific test outcome. The test can be self-administered after an interactive online teaching session and does not require a visit to a health care facility or use of a medical lab. To address these goals, we can take advantage of several CGM data sets and technologies available at the UVA Center for Diabetes Technology including: (i) A large collection of daily CGM profiles from healthy, type 1 diabetes, and type 2 diabetes individuals, (ii) simultaneously collected OGTT and home CGM data, and (iii) in silico technology to simulate the progression of individual's daily CGM profiles and responses to an OGTT and CGM/SLMM from health to full-blown type 1 or type 2 diabetes.

### Background and Significance

Early screening for identifying individuals at increased diabetes risk can reduce the rates of complications at diagnosis, improve the post-diagnosis quality of glycemic control, potentially slow the progression, reduce future poor health outcomes and diabetes-related complications. Predicting the risk for developing diabetes however poses a significant challenge and many efforts have been devoted to develop simple screening tests, including metabolic and genetic tests. Of significant clinical relevance is these tests to be cheap, easy to administer, provide as much information as possible, and present minimal burden to the individual. Ideally, as proposed in this application, they should be self-administered without the need for a visit to a clinical facility or use of a clinical lab. They should also estimate not only the life-long risk for an individual to develop diabetes, but should be also able to assess the state of progression and the short-term risk for imminent diagnosis. For example, screening for genetic diabetes risk (both type 1 and type 2) can be performed as an at-home test. However, this test cannot account for the level of progression towards development of full-blown diabetes. As another example, the Oral Glucose Tolerance Test (OGTT) can be used for diagnosis of diabetes and estimating the level of glucose tolerance impairment. However, an OGTT requires a visit to a health care facility and use of a medical lab. Recently, we have proposed a novel technology for estimating the immunological risk for type 1 diabetes (presence or absence of insulin autoantibodies) which uses a self-administered one-week home Continuous Glucose Monitoring (CGM) test with tree standardized liquid mixed meals tests (SLMM) taken in lieu of three breakfasts and combined with dedicated machine-learning (ML)-based analysis of the collected data. We have shown that the CGM/SLMM/ML technology captures subtle differences in glycemia in the very early stages of the disease and relates them to the individual's immunological risk for type 1 diabetes. The technology however, is not limited to detection of immunological risk for T1D only. In this application, we contemplate use of similar test technologies, which combine the one-week CGM/SLMM home data collection test with dedicated, test-specific, ML-based analyses for diagnosis and risk assessment of T1D, T2D, and distinguishing between T1D and T2D.

The research takes advantage of the apparent sensitivity of the CGM/SLMM home test to subtle changes in glycemia. Our specific aims are formulated as three milestones, each addressing a new CGM-based test technology. Common to all tests is the CGM/SLMM at-home data collection protocol. The tests differ in the specific ML methodology for data analysis required for addressing each clinical objective.
Milestone 1 [M1]. CGM-based OGTT replacement for diagnosis of diabetes and other metabolic disorders and assessment of the level of impairment of the glucose tolerance.
Milestone 2 [M2]. Diagnosis of diabetes with stratification between T1 and T2 diabetes.
Milestone 3 [M3]. Assessing the risk and level of progression (time course) of an individual towards overt disease type 1 or type 2 diabetes

We take advantage of several unique to our Center for Diabetes Technology data sets and capabilities:
(a) Simultaneous hospital OGTT and home CGM/SLMM test data in healthy individuals (M1);
(b) A large CGM data set from healthy individuals, and from T1 and T2 diabetes patients (M2 & M3);
(c) Ability to generate virtual CGM/SLMM data representative for the progression from health to full-blown type 1 or type 2 diabetes with accounting for the time course of the process (M3).

### Technology Framework

### Diagnosis and Screening for Diabetes

Early identification and screening of individuals at increased diabetes risk can reduce the rates of diabetic ketoacidosis (DKA) at diagnosis (e.g., for T1D), improve the quality of glycemic control, reduce future poor health outcomes and complications (T1D and T2D). There are several clinical tests to diagnose diabetes (https://diabetes.org/diabetes/a1c/diagnosis). These include blood tests for HbA1C, fasting plasma glucose, 2-hour OGTT, random plasma glucose tests, etc. In addition, diagnosis of type 1 diabetes requires follow-up tests for islet autoantibodies and/or ketones in the urine. Notably, these tests require a visit to a health care facility and use of a clinical laboratory. Screening for diabetes in healthy individuals can be performed by an islet auto-antibody tests (T1D) or genetic tests (T1D and T2D) at home, with some recent attempts to use CGM as an alternative or as a complementary test (below).

### Screening for Gestational Diabetes Mellitus (GDM)

Women who are at average risk of GDM (overweight and with family history of diabetes) are currently recommended an OGTT between 24 and 28 gestational weeks as the method of GDM diagnosis. The proposed here OGTT replacement can reduce the burden of the OGTT data collection. As described in the section “Funding plan for after award expiration” we envision the verification of the new OGTT replacement technology in a clinical trial in pregnant women as an immediate next step.

### Published Data on CGM Use for Assessment of Diabetes Risk

Several studies employed CGM not only in people with diabetes but also in obese individuals and in individuals at different stages of prediabetes. Studies have suggested that CGM can be used for detecting early hyperglycemia in children with multiple autoantibodies, and for predicting progression to diabetes in antibody positive (Ab+) children. In, the authors suggested that “CGM should be included in the ongoing monitoring of high-risk children (Ab+)”. In addition, in T2D, CGM was able to detect impaired glycemia earlier than other standard biomarkers used for diagnosis and classification. As disclosed herein, a one-week CGM test has been investigated for its ability to identify individuals at higher risk for rapid progression to Stage 3 T1D (full-blown disease) and had identified several CGM-derived metrics of hyperglycemia associated with progression to Stage 3.

It should be noted that existing tests either define a general timeframe of disease progression (e.g., genetic or auto-antibody tests) or, as with CGM tests, are sensitive only to imminent progression to diabetes. Compared to existing CGM tests, our data collection strategy is the only one that uses SLMM to provide additional, more controlled, data from the home CGM test to enhance its sensitivity and predictive capabilities.

### Product Description

It is contemplated to develop several CGM-based tests technologies which use one and the same data collection protocol to replace or augment existing mainstream tests for diagnosis of T1 or T2 diabetes, gestational diabetes, metabolic syndrome, or other conditions associated with abnormalities in glucose homeostasis, occurring early in the course of the disease. These tests have the following advantages:
(i) Can be self-administered at home after an interactive online session
(ii) Does not require a visit to a health care facility, blood sample collection, or use of a clinical laboratory
(iii) A one-week CGM/SLMM data collection provides data for several tests for diagnosis and risk assessment.

All tests include a data collection phase and a data analysis phase. The data collection phase is the same for all tests and consists of a seven-day home CGM data collection during which the tested individual consumes three SLMM (Boost) instead of three breakfasts. It can be self-administered after an interactive online training session. The data is collected remotely are analyzed with a test-specific ML methodology during the data analysis phase.

### Research Plan

Below, we describe the CGM/SLMM home data collection test, common for all test technologies. Second, we outline our most current results in using the CGM/SLMM to define a technology for predicting the T1D immunological risk for Stage 1 and 2 prediabetes. Third, we describe the CDT resources available this project. Finally, we address each of the Milestones.

The design of the CGM protocol is as follows. Individuals are asked to wear a CGM for 7 days according to the manufacturer recommendations and are instructed to return study supplies after the end of the study. During the 7-day period of CGM wearing, participants are asked to have three identical mixed meal caloric drinks [SLMM] (Boost; provided by our team) at 8 am on Day 3, 5, and 7 instead of three breakfasts. Two hours after the second Boost, participants are asked to take a urine sample to be mailed back to the study team for evaluating the individual's C-peptide responses to the Boost via measuring the C-peptide/creatinine ratio. Participants are also asked to record the timing of the SLMMs during the one-week period of wearing the CGM. It should be noted that the CGM/SLMM home data collection can be self-administered after an interactive online teaching session and does not require a visit to a health care facility or use of a medical lab.

### Preliminary Results

Sixty healthy relatives of people with T1D with mean±SD age of 23.7±10.7 years, HbA1c of 5.3±0.3%, and BMI of 23.8±5.6 kg/m2 with zero (N=21), one (N=18), and ≥2 (N=21) islet autoantibodies, were enrolled in an NIH TrialNet ancillary study. Participants wore a CGM for a week and consumed three standardized liquid mixed meals (SLMM) instead of three breakfasts (above). Glycemic outcomes were computed from weekly, overnight (12:00-06:00), and post-SLMM CGM traces, compared across groups, and used in four supervised machine-learning autoantibody status classifiers. Classifiers were evaluated via 10-fold cross-validation using the receiver operating characteristic area under the curve (AUC-ROC) to select the best classification model. Among all computed glycemia metrics, only three were different across the autoantibodies groups: percent time >180 mg/dL (T180) weekly (p=0.04), overnight CGM incremental AUC (p=0.005), and T180 for 75 min post-SLMM CGM traces (p=0.004). Once overnight and post-SLMM features were incorporated in the machine-learning classifiers, a linear Support Vector Machine (SVM) model achieved the best performance of classifying antibody positive vs. antibody negative participants with AUC-ROC ≥0.81. This demonstrates the advantage of adding SLMM to the one-week CGM test. In this study we did not have data to distinguish progressors from non-progressors and to test whether our methodology can predict the time to T1D diagnosis. These data have been requested from TrialNet. As shown recently, a simple CGM test (without SLMM and ML) can be useful for identifying individuals at higher risk for rapid progression to Stage 3 T1D. Based on our results, we expect our technology to be more sensitive and to perform better. In conclusion, a new technology combining machine learning with a potentially self-administered one-week CGM home test can help improve T1D risk detection without the need to visit a hospital or use a lab.

Most relevant to the current application is the finding that dedicated machine learning methods and addition of SLMM to the CGM data collection led to technology that is sensitive to subtle changes in glycemia very early in the pathogenesis of the disease. Traditional method cannot achieve such level of sensitivity. If compared to conventional auto-antibody or genetic tests, which can be done at home as well (but require a medical lab), CGM-based tests provide the opportunity to evaluate the level of glucose intolerance, which can complement other diabetes risk biomarkers, to better estimate the overall risk of diabetes, separate progressors from non-progressors, and predict the time course to overt disease.

Available Data for this Project

The following datasets are available at the CDT:
(1) The already described TrialNet dataset: CGM/SLMM in 60 healthy subjects plus an OGTT. This data set is unique and will allow us to address M1.
(2) A large dataset with CGM traces from healthy individuals, and from type 1 and type 2 patients. This set, along with the simulation technology described below will be used for addressing M2 and M3.

### CDT Simulation Technology

CDT has a computer-based simulating technology to create virtual subjects spanning the progression from normal glucose metabolism to T1 or T2 diabetes. This will allow us to address M2 and M3.

We will use one common approach to all tree milestones as described below.

1. We start by splitting the available 7-day CGM/SLMM test data (real or virtual) into three components:
a. “Weekly” data set is the complete 7-day CGM trace
b. “Overnight” includes all 7-day overnight periods (12:00-06:00)
c. “PostSLMM” which include only on the 75 to 120 min of the post-SLMM CGM.

2. Multiple glycemic outcomes are computed from the weekly, overnight, and post-SLMM CGM traces. These include, mean glucose, percent time in different glycemic ranges, glucose variability metrics, various risk measures, glucose area under the curve, and metrics specific to the post-SLMM periods only (e.g., maximal glucose amplitude, time to maximal glucose, slope of post-SLMM glucose increase, etc.)

3. These metrics are used with different classification models to develop, milestone specific classifiers and define the best classifier model. We will use at least four models: linear discriminant analysis (LDA), linear support vector machine (SVM), logistic regression (LR), and K-nearest neighbors (KNN).

4. The three milestones are addressed as follows:
a. M1. We will classify and predict traditional OGTT based outcomes, like for example the 2-hour glucose concentration after the glucose load.
b. M2 and M3 will require generation of virtual data with our simulation environment. We will use the available real dataset (above) to simulate OGTT and CGM/SLMM responses starting from a healthy metabolism up to diagnosis of full-blown type 1 or type 2 diabetes.
c. M2. We will select the best classifier model to distinguish T1D from T2D at the time of diagnosis.
d. M3. We will define the earliest time at which the classifiers can establish a significantly elevated risk for an individual to develop T1D od T2D and will establish conditions under which the technology can distinguish T1D from T2D risk.

### Exemplary System and Device Configurations

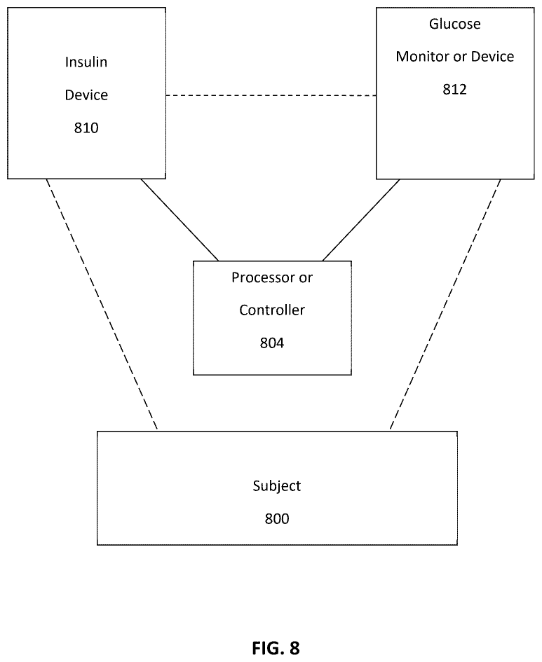

FIG. 8 is an exemplary high-level functional block diagram for an embodiment of the present invention, or an aspect of an embodiment of the present invention. As shown in FIG. 8, a processor 804 or controller communicates with the glucose monitor or data source 812, and optionally an insulin delivery device (e.g., other device 810). The glucose monitor or device communicates with the subject 800 to monitor glucose levels of the subject 800. The processor 804 or controller is configured to perform the required calculations. Optionally, the insulin delivery device communicates with the subject 800 to deliver insulin to the subject 800. The processor 804 or controller is configured to perform the required calculations. The glucose monitor and the insulin delivery device may be implemented as a separate device or as a single device. The processor 804 can be implemented locally in the glucose monitor, the insulin delivery device, or a standalone device (or in any combination of two or more of the glucose monitor, insulin device, or a stand along device). The processor 804 or a portion of the system can be located remotely such that the device is operated as a telemedicine device.

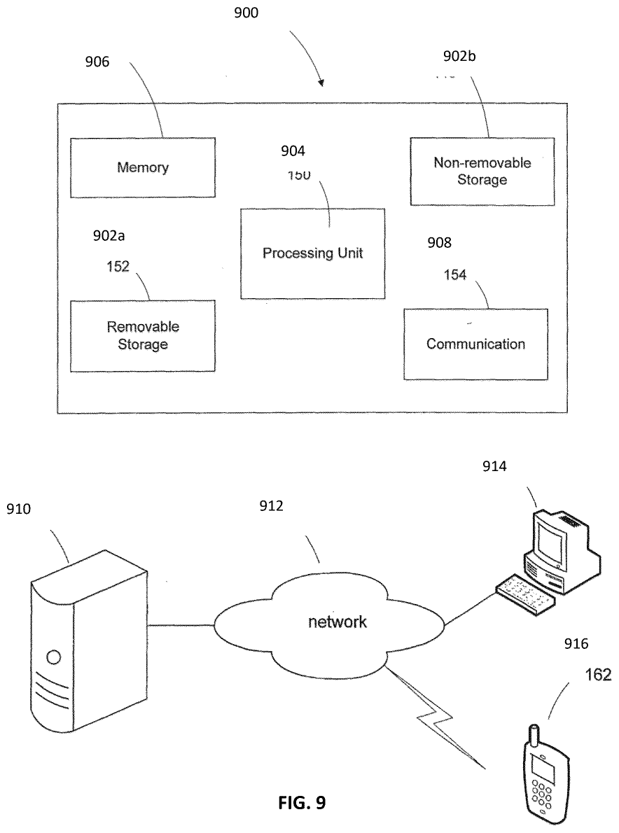

Referring to FIG. 9, in its most basic configuration, computing device 900 typically includes at least one processor 904 and memory 906. Depending on the exact configuration and type of computing device, memory 906 can be volatile (such as RAM), non-volatile (such as ROM, flash memory, etc.) or some combination of the two.

Additionally, the computing device 900 may also have other features and/or functionality. For example, the computing device 900 could also include additional removable and/or non-removable storage including, but not limited to, magnetic or optical disks or tape, as well as writable electrical storage media. Such additional storage is the figure by removable storage 902a and non-removable storage 902b. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, the device.

The computing device 900 may also contain one or more communications connections 908 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode, execute, or process information in the signal. By way of example, and not limitation, communication medium includes wired media such as a wired network or direct-wired connection, and wireless media such as radio, RF, infrared and other wireless media. As discussed above, the term computer readable media as used herein includes both storage media and communication media.

In addition to a stand-alone computing machine, embodiments of the invention can also be implemented on a network system comprising a plurality of computing devices that are in communication with a networking means, such as a network with an infrastructure or an ad hoc network. The network connection can be wired connections or wireless connections. As a way of example, FIG. 9 illustrates a network system in which embodiments of the invention can be implemented. In this example, the network system comprises computer 910 (e.g. a network server), network connection means 912 (e.g. wired and/or wireless connections), computer terminal 914, and PDA (e.g. a smart-phone) 916 (or other handheld or portable device, such as a cell phone, laptop computer, tablet computer, GPS receiver, mp3 player, handheld video player, pocket projector, etc. or handheld devices (or non portable devices) with combinations of such features). In an embodiment, it should be appreciated that the module 914 may be glucose monitor device. In an embodiment, it should be appreciated that the module listed as 914 may be a glucose monitor device, artificial pancreas, and/or an insulin device (or other interventional or diagnostic device). Any of the components may be multiple in number. The embodiments of the invention can be implemented in anyone of the devices of the system. For example, execution of the instructions or other desired processing can be performed on the same computing device 900. Alternatively, an embodiment of the invention can be performed on different computing devices of the network system. For example, certain desired or required processing or execution can be performed on one of the computing devices of the network (e.g., server 910 and/or glucose monitor device), whereas other processing and execution of the instruction can be performed at another computing device (e.g., terminal 914) of the network system, or vice versa. In fact, certain processing or execution can be performed at one computing device (e.g. server 910 and/or insulin device, artificial pancreas, or glucose monitor device (or other interventional or diagnostic device)); and the other processing or execution of the instructions can be performed at different computing devices that may or may not be networked. For example, the certain processing can be performed at terminal 914, while the other processing or instructions are passed to a computing device 900 where the instructions are executed. This scenario may be of particular value especially when the PDA device, for example, accesses to the network through computer terminal 914 (or an access point in an ad hoc network). For another example, software to be protected can be executed, encoded or processed with one or more embodiments of the invention. The processed, encoded or executed software can then be distributed to customers. The distribution can be in a form of storage media (e.g., disk) or electronic copy.

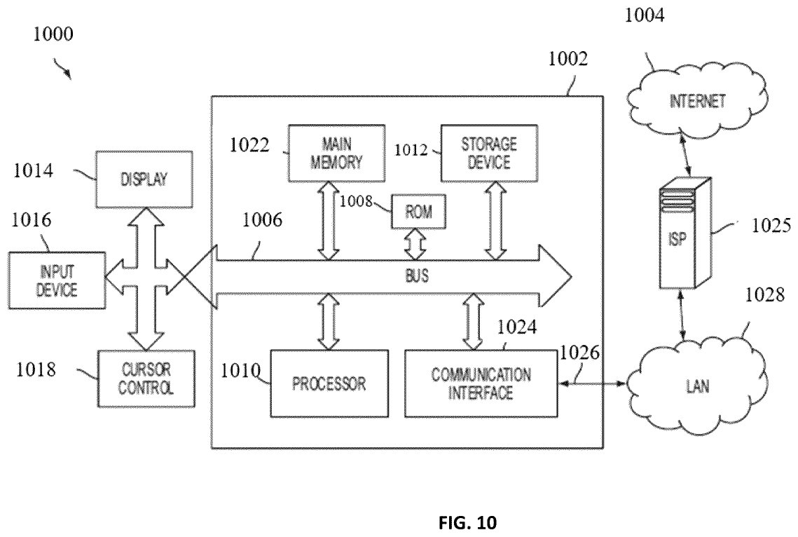

FIG. 10 is a block diagram that illustrates a system 1000 including a computer system 1002 and the associated Internet 1004 connection upon which an embodiment may be implemented. Such configuration is typically used for computers (hosts) connected to the Internet 1004 and executing a server or a client (or a combination) software. A source computer such as laptop, an ultimate destination computer and relay servers, for example, as well as any computer or processor described herein, may use the computer system configuration and the Internet connection shown in FIG. 10. The system 1004 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 10 illustrates various components of a computer system, it is not intended to represent any particular architecture or manner of interconnecting the components; as such details are not germane to the present invention. It will also be appreciated that network computers, handheld computers, cell phones and other data processing systems which have fewer components or perhaps more components may also be used. The computer system of FIG. 10 may, for example, be an Apple Macintosh computer or Power Book, or an IBM compatible PC. Computer system 1000 includes a bus 1006, an interconnect, or other communication mechanism for communicating information, and a processor 110, commonly in the form of an integrated circuit, coupled with bus 1006 for processing information and for executing the computer executable instructions. Computer system 1000 also includes a main memory 1008, such as a Random Access Memory (RAM) or other dynamic storage device, coupled to bus 1006 for storing information and instructions to be executed by processor 1010.

Main memory 1008 also may be used for storing temporary variables or other intermediate information during execution of instructions to be executed by processor 1010. Computer system 1000 further includes a Read Only Memory (ROM) 1008 (or other non-volatile memory) or other static storage device coupled to bus 1006 for storing static information and instructions for processor 1010. A storage device 1012, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 1006 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 1000 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 1000 may be coupled via bus 1006 to a display 1014, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 1016, including alphanumeric and other keys, is coupled to bus 1006 for communicating information and command selections to processor 1010. Another type of user input device is cursor control 1018, such as a mouse, a trackball, or cursor direction keys for communicating direction information and command selections to processor 1010 and for controlling cursor movement on display 1014. This input device typically has two degrees of freedom in two axes, a first axis (e.g., x) and a second axis (e.g., y), that allows the device to specify positions in a plane.

The computer system 1002 may be used for implementing the methods and techniques described herein. According to one embodiment, those methods and techniques are performed by computer system 1002 in response to processor 1010 executing one or more sequences of one or more instructions contained in main memory 1020. Such instructions may be read into main memory 1022 from another computer-readable medium, such as storage device 1012. Execution of the sequences of instructions contained in main memory 1022 causes processor 1010 to perform the process steps described herein. In alternative embodiments, hard-wired circuitry may be used in place of or in combination with software instructions to implement the arrangement. Thus, embodiments of the invention are not limited to any specific combination of hardware circuitry and software.

The term “computer-readable medium” (or “machine-readable medium”) as used herein is an extensible term that refers to any medium or any memory, that participates in providing instructions to a processor, (such as processor 1010) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 1006. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 1010 for execution. For example, the instructions may initially be carried on a magnetic disk of a remote computer. The remote computer can load the instructions into its dynamic memory and send the instructions over a telephone line using a modem. A modem local to computer system 1000 can receive the data on the telephone line and use an infra-red transmitter to convert the data to an infra-red signal. An infra-red detector can receive the data carried in the infra-red signal and appropriate circuitry can place the data on bus 1006. Bus 1006 carries the data to main memory 1022, from which processor 1010 retrieves and executes the instructions. The instructions received by main memory 1022 may optionally be stored on storage device 1012 either before or after execution by processor 1010.

Computer system 1000 also includes a communication interface 1024 coupled to bus 1006. Communication interface 1024 provides a two-way data communication coupling to a network link 1026 that is connected to a local network 1028. For example, communication interface 1024 may be an Integrated Services Digital Network (ISDN) card or a modem to provide a data communication connection to a corresponding type of telephone line. As another non-limiting example, communication interface 1024 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 1818 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Feb. 20, 2004), which is incorporated in its entirety for all purposes as if fully set forth herein.

Wireless links may also be implemented. In any such implementation, communication interface 1024 sends and receives electrical, electromagnetic or optical signals that carry digital data streams representing various types of information.

Network link 1026 typically provides data communication through one or more networks to other data devices. For example, network link 1026 may provide a connection through local network 1028 to a host computer or to data equipment operated by an Internet Service Provider (ISP) 1025. ISP 1025 in turn provides data communication services through the world wide packet data communication network Internet 1004. Local network 1028 and Internet 1004 both use electrical, electromagnetic or optical signals that carry digital data streams. The signals through the various networks and the signals on the network link 1026 and through the communication interface 1024, which carry the digital data to and from computer system 1000, are exemplary forms of carrier waves transporting the information.

A received code may be executed by processor 1010 as it is received, and/or stored in storage device 1012, or other non-volatile storage for later execution. In this manner, computer system 1000 may obtain application code in the form of a carrier wave.

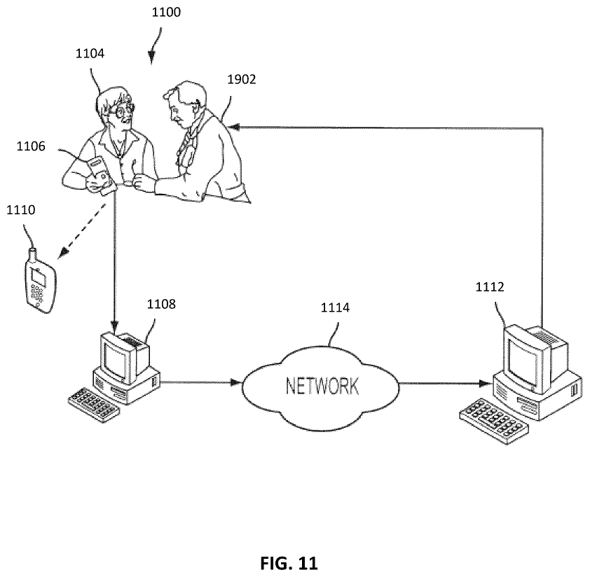

FIG. 11 illustrates a system in which one or more embodiments of the invention can be implemented using a network, or portions of a network or computers. Although the present invention glucose monitor, artificial pancreas or insulin device (or other interventional or diagnostic device) may be practiced without a network. FIG. 11 diagrammatically illustrates an exemplary system in which examples of the invention can be implemented. In an embodiment the glucose monitor, artificial pancreas or insulin device (or other interventional or diagnostic device) may be implemented by the subject (or patient) locally at home or other desired location. However, in an alternative embodiment it may be implemented in a clinic setting or assistance setting. For instance, a clinic setup 1100 provides a place for doctors (e.g. 1102) or clinician/assistant to diagnose patients (e.g. 1104) with diseases related with glucose and related diseases and conditions. A glucose monitoring device 1106 can be used to monitor and/or test the glucose levels of the patient—as a standalone device. It should be appreciated that while only glucose monitor device 1106 is shown in the figure, the system of the invention and any component thereof may be used in the manner depicted by FIG. 11. The system or component may be affixed to the patient or in communication with the patient as desired or required. For example the system or combination of components thereof—including a glucose monitor device 1106 (or other related devices or systems such as a controller, and/or an artificial pancreas, an insulin pump (or other interventional or diagnostic device), or any other desired or required devices or components)—may be in contact, communication or affixed to the patient through tape or tubing (or other medical instruments or components) or may be in communication through wired or wireless connections. Such monitor and/or test can be short term (e.g. clinical visit) or long term (e.g. clinical stay or family). The glucose monitoring device outputs can be used by the doctor (clinician or assistant) for appropriate actions, such as insulin injection or food feeding for the patient, or other appropriate actions or modeling. Alternatively, the glucose monitoring device output can be delivered to computer terminal 1112 for instant or future analyses. The delivery can be through cable or wireless or any other suitable medium. The glucose monitoring device output from the patient can also be delivered to a portable device, such as PDA 1110. The glucose monitoring device outputs with improved accuracy can be delivered to a glucose monitoring center 1112 for processing and/or analyzing. Such delivery can be accomplished in many ways, such as network connection 1114, which can be wired or wireless.

In addition to the glucose monitoring device outputs, errors, parameters for accuracy improvements, and any accuracy related information can be delivered, such as to computer and/or glucose monitoring center 1112 for performing error analyses. This can provide a centralized accuracy monitoring, modeling and/or accuracy enhancement for glucose centers (or other interventional or diagnostic centers), due to the importance of the glucose sensors (or other interventional or diagnostic sensors or devices).

Examples of the invention can also be implemented in a standalone computing device associated with the target glucose monitoring device, artificial pancreas, and/or insulin device (or other interventional or diagnostic device.

FIG. 12 is a block diagram illustrating an example of a machine upon which one or more aspects of embodiments of the present invention can be implemented. Referring to FIG. 12, an aspect of an embodiment of the present invention includes, but not limited thereto, a system, method, and computer readable medium, which illustrates a block diagram of an example machine 1200 upon which one or more embodiments (e.g., discussed methodologies) can be implemented (e.g., run).

FIG. 12 illustrates a block diagram of an example machine 1200 upon which one or more embodiments (e.g., discussed methodologies) can be implemented (e.g., run).

Examples of machine 1200 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).).

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 1200) and software architectures that can be deployed in example embodiments.

In an example, the machine 1200 can operate as a standalone device or the machine 1200 can be connected (e.g., networked) to other machines.

In a networked deployment, the machine 1200 can operate in the capacity of either a server or a client machine in server-client network environments. In an example, machine 1200 can act as a peer machine in peer-to-peer (or other distributed) network environments. The machine 1200 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 1200. Further, while only a single machine 1200 is illustrated, the term “machine” shall also be taken to include any collection of machines that individually or jointly execute a set (or multiple sets) of instructions to perform any one or more of the methodologies discussed herein.

Example machine (e.g., computer system) 1200 can include a processor 1250 (e.g., a central processing unit (CPU), a graphics processing unit (GPU) or both), a main memory 1252a and a static memory 1252b, some or all of which can communicate with each other via a bus 1220. The machine 1200 can further include a display unit 1202, an alphanumeric input device 1204 (e.g., a keyboard), and a user interface (UI) navigation device 1206 (e.g., a mouse). In an example, the display unit 1202, input device 1204 and UI navigation device 1206 can be a touch screen display. The machine 1200 can additionally include a storage device (e.g., drive unit) 1208, a signal generation device 1210 (e.g., a speaker), a network interface device 1212, and one or more sensors 1214, such as a global positioning system (GPS) sensor, compass, accelerometer, or other sensor.

The storage device 1208 can include a machine readable medium 1216 on which is stored one or more sets of data structures or instructions 1254 (e.g., software) embodying or utilized by any one or more of the methodologies or functions described herein. The instructions 1254 can also reside, completely or at least partially, within the main memory 1252a, within static memory 1252b, or within the processor 804 during execution thereof by the machine 1200. In an example, one or any combination of the processor 804, the main memory 1252a, the static memory 1252b, or the storage device 1208 can constitute machine readable media.

While the machine readable medium 1216 is illustrated as a single medium, the term “machine readable medium” can include a single medium or multiple media (e.g., a centralized or distributed database, and/or associated caches and servers) that configured to store the one or more instructions 1254. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 1254 can further be transmitted or received over a communications network 1218 using a transmission medium via the network interface device utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the invention described herein.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

### REFERENCES

The following references listed below and throughout this document are hereby incorporated by reference in their entirety herein, and which are not admitted to be prior art with respect to the present invention by inclusion in this section.
Shafiei M, Farhy L, Lobo B, Kovatchev B. Classification of Daily Continuous Glucose Monitoring (CGM) Profiles in Type 1 Diabetes Using Layered Clustering and Clinical Metrics; 2020 Diabetes Technology Meeting. JDST 15(2) Mar. 2, 2021.
Montaser E, Villard O, Fabris C, Lobo B, Brown S, DeBoer M, Kovatchev B, Farhy L. Detecting the Risk of Type 1 Diabetes Through Clustering of Data Collected During a Self-Administered CGM-based Home Test. Accepted at ATTD 2022, Barcelona, Spai
Wang K, Farhi L, Kovatchev B. International Patent Application Serial No. PCT/US2020/060366, entitled “SYSTEM, METHOD AND COMPUTER READABLE MEDIUM FOR COMPRESSING CONTINUOUS GLUCOSE MONITOR DATA”, filed Nov. 13, 2020; Publication No. WO 2021/097178, May 20, 2021.
U.S. Utility patent application Ser. No. 17/776,717, entitled “METHOD AND SYSTEM FOR MODEL-BASED TRACKING OF HEMOGLOBIN A1c FROM DAILY CONTINUOUS GLUCOSE MONITORING PROFILES”, filed May 13, 2022.
International Patent Application Serial No. PCT/US2020/060658, entitled “METHOD AND SYSTEM FOR MODEL-BASED TRACKING OF HEMOGLOBIN A1c FROM DAILY CONTINUOUS GLUCOSE MONITORING PROFILES”, filed Nov. 16, 2020; Publication No. WO 2021/097396, May 20, 2021.
U.S. Utility patent application Ser. No. 17/776,056, entitled “SYSTEM, METHOD AND COMPUTER READABLE MEDIUM FOR COMPRESSING CONTINUOUS GLUCOSE MONITOR DATA”, filed May 11, 2022.
International Patent Application Serial No. PCT/US2020/060366, entitled “SYSTEM, METHOD AND COMPUTER READABLE MEDIUM FOR COMPRESSING CONTINUOUS GLUCOSE MONITOR DATA”, filed Nov. 13, 2020; Publication No. WO 2021/097178, May 20, 2021.
U.S. Utility patent application Ser. No. 17/694,870, entitled “INSULIN MONITORING AND DELIVERY SYSTEM AND METHOD FOR CGM BASED FAULT DETECTION AND MITIGATION VIA METABOLIC STATE TRACKING”, filed Mar. 15, 2022.
U.S. Utility patent application Ser. No. 17/683,676, entitled “System, Method and Computer Readable Medium for Dynamical Tracking of the Risk for Hypoglycemia in Type 1 and Type 2 Diabetes”, filed Mar. 1, 2022.
U.S. Utility patent application Ser. No. 15/958,257, entitled “System, Method and Computer Readable Medium for Dynamical Tracking of the Risk for Hypoglycemia in Type 1 and Type 2 Diabetes”, filed Apr. 20, 2018; U.S. Pat. No. 11,289,201, issued Mar. 29, 2022.
International Patent Application Serial No. PCT/US2016/058234, entitled “System, Method and Computer Readable Medium for Dynamical Tracking of the Risk for Hypoglycemia in Type 1 and Type 2 Diabetes”, filed Oct. 21, 2016; Publication No. WO 2017/070553, Apr. 27, 2017.
International Patent Application Serial No. PCT/US2022/017489, entitled “METHOD AND SYSTEM FOR QUANTITATIVE PHYSIOLOGICAL ASSESSMENT AND PREDICTION OF CLINICAL SUBTYPES OF GLUCOSE METABOLISM DISORDERS”, filed Feb. 23, 2022.
International Patent Application Serial No. PCT/US2022/017449, entitled “METHOD AND SYSTEM FOR MAPPING INDIVIDUALIZED METABOLIC PHENOTYPE TO A DATABASE IMAGE FOR OPTIMIZING CONTROL OF CHRONIC METABOLIC CONDITIONS”, filed Feb. 23, 2022.
U.S. Utility patent application Ser. No. 17/590,659, entitled “System, Method, and Computer Simulation Environment for In Silico Trials in Pre-Diabetes and Type 2 Diabetes”, filed Feb. 1, 2022.
U.S. Utility patent application Ser. No. 13/380,839, entitled “System, Method, and Computer Simulation Environment for In Silico Trials in Pre-Diabetes and Type 2 Diabetes”, filed Dec. 25, 2011; U.S. Pat. No. 11,238,990, issued Feb. 1, 2022.
International Patent Application Serial No. PCT/US2010/040097, entitled “System, Method, and Computer Simulation Environment for In Silico Trials in Prediabetes and Type 2 Diabetes”, filed Jun. 25, 2010; Publication No. WO 2010/151834, Dec. 29, 2010.
International Patent Application Serial No. PCT/US2021/045936, entitled “METHOD AND SYSTEM FOR GENERATING A USER TUNABLE REPRESENTATION OF GLUCOSE HOMEOSTASIS IN TYPE 1 DIABETES BASED ON AUTOMATED RECEIPT OF THERAPY PROFILE DATA”, filed Aug. 13, 2021; Publication No. WO 2022/036214, Feb. 17, 2022.
U.S. Utility patent application Ser. No. 17/339,153, entitled “Method and System for the Safety, Analysis, and Supervision of Insulin Pump Action and Other Modes of Insulin Delivery in Diabetes”, filed Jun. 4, 2021; Publication No. US 2021-0313036 A1, Oct. 7, 2021.
U.S. Utility patent application Ser. No. 13/634,040, entitled “Method and System for the Safety, Analysis, and Supervision of Insulin Pump Action and Other Modes of Insulin Delivery in Diabetes”, filed Sep. 11, 2012; U.S. Pat. No. 11,069,434, issued Jul. 20, 2021.
International Patent Application Serial No. PCT/US2011/028163, entitled “Method and System for the Safety, Analysis, and Supervision of Insulin Pump Action and Other Modes of Insulin Delivery in Diabetes”, filed Mar. 11, 2011; Publication No. WO 2011/112974, Sep. 15, 2011.
U.S. Utility patent application Ser. No. 17/333,161, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed May 28, 2021; Publication No. US 2021-0282677 A1, Sep. 16, 2021.
U.S. Utility patent application Ser. No. 17/070,245, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed Oct. 14, 2020; Publication No. US 2021/0038132 A1, Feb. 11, 2021.
U.S. Utility patent application Ser. No. 15/669,111, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed Aug. 4, 2017; U.S. Pat. No. 10,842,419, issued Nov. 24, 2020.
U.S. Utility patent application Ser. No. 14/015,831, entitled “CGM-Based Prevention of Hypoglycemia Via Hypoglycemia Risk Assessment and Smooth Reduction of Insulin Delivery”, filed Aug. 30, 2013; U.S. Pat. No. 9,750,438, issued Sep. 5, 2017.
U.S. Utility patent application Ser. No. 13/203,469, entitled “CGM-Based Prevention of Hypoglycemia via Hypoglycemia Risk Assessment and Smooth Reduction Insulin Delivery”, filed Aug. 25, 2011; U.S. Pat. No. 8,562,587, issued Oct. 22, 2013.
International Patent Application Serial No. PCT/US2010/025405, entitled “CGM-BASED PREVENTION OF HYPOGLYCEMIA VIA HYPOGLYCEMIA RISK ASSESMENT AND SMOOTH REDUCTION INSULIN DELIVERY”, filed Feb. 25, 2010; Publication No. WO 2010/099313 A1, Sep. 2, 2010.
U.S. Utility patent application Ser. No. 17/181,888, entitled “SYSTEMS OF CENTRALIZED DATA EXCHANGE FOR MONITORING AND CONTROL OF BLOOD GLUCOSE”, filed Feb. 22, 2021; Publication No. US 2021-0169409 A1, Jun. 10, 2021.
U.S. Utility patent application Ser. No. 15/109,682, entitled “SYSTEMS OF CENTRALIZED DATA EXCHANGE FOR MONITORING AND CONTROL OF BLOOD GLUCOSE”, filed Jul. 5, 2016; U.S. Pat. No. 10,925,536, issued Feb. 23, 2021.
International Patent Application Serial No. PCT/US2015/010167, entitled “Central Data Exchange Node For System Monitoring and Control of Blood Glucose Levels in Diabetic Patients”, filed Jan. 5, 2015; Publication No. WO2015103543, Jul. 9, 2015.
U.S. Utility patent application Ser. No. 17/156,169, entitled “Method, System and Computer Program Product for Real-Time Detection of Sensitivity Decline in Analyte Sensors”, filed Jan. 22, 2021; Publication No. US 2021-0218481 A1, Jul. 15, 2021.
U.S. Utility patent application Ser. No. 15/866,384, entitled “Method, System and Computer Program Product for Real-Time Detection of Sensitivity Decline in Analyte Sensors”, filed Jan. 9, 2018; U.S. Pat. No. 10,903,914, issued Jan. 26, 2021.
U.S. Utility patent application Ser. No. 14/266,612, entitled “Method, System and Computer Program Product for Real-Time Detection of Sensitivity Decline in Analyte Sensors”, filed Apr. 30, 2014; U.S. Pat. No. 9,882,660, issued Jan. 30, 2018.
U.S. Utility patent application Ser. No. 13/418,305, entitled “Method, System and Computer Program Product for Real-Time Detection of Sensitivity Decline in Analyte Sensors”, filed Mar. 12, 2012; U.S. Pat. No. 8,718,958, issued May 6, 2014.
International Patent Application Serial No. PCT/US2007/082744, entitled “Method, System and Computer Program Product for Real-Time Detection of Sensitivity Decline in Analyte Sensors”, filed Oct. 26, 2007; Publication No. WO/2008/052199, May 2, 2008.
U.S. Utility patent application Ser. No. 11/925,689, entitled “Method, System and Computer Program Product for Real-Time Detection of Sensitivity Decline in Analyte Sensors”, filed Oct. 26, 2007; U.S. Pat. No. 8,135,548, issued Mar. 13, 2012.
U.S. Utility patent application Ser. No. 17/132,604, entitled “ACCURACY CONTINUOUS GLUCOSE MONITORING METHOD, SYSTEM, AND DEVICE”, filed Dec. 23, 2020; Publication No. US 2021-0113122 A1, Apr. 22, 2021.
U.S. Utility patent application Ser. No. 15/510,878, entitled “ACCURACY CONTINUOUS GLUCOSE MONITORING METHOD, SYSTEM, AND DEVICE”, filed Mar. 13, 2017; U.S. Pat. No. 10,881,334, issued Jan. 5, 2021.
International Patent Application Serial No. PCT/US2015/045340, entitled “IMPROVED ACCURACY CONTINUOUS GLUCOSE MONITORING METHOD, SYSTEM, AND DEVICE”, filed Aug. 14, 2015; Publication No. WO2016025874, Feb. 18, 2016.
U.S. Utility patent application Ser. No. 16/789,901, entitled “Unified Platform For Monitoring and Control of Blood Glucose Levels in Diabetic Patients”, filed Feb. 13, 2020; Publication No. US-2020-0214629-A1, Jul. 9, 2020.
U.S. Utility patent application Ser. No. 14/128,922, entitled “Unified Platform For Monitoring and Control of Blood Glucose Levels in Diabetic Patients”, filed Dec. 23, 2013; U.S. Pat. No. 10,610,154, issued Apr. 7, 2020.
International Patent Application Serial No. PCT/US2012/043910, entitled “Unified Platform For Monitoring and Control of Blood Glucose Levels in Diabetic Patients”, filed Jun. 23, 2012; Publication No. WO 2012/178134, Dec. 27, 2012.
U.S. Utility patent application Ser. No. 16/588,881, entitled “Tracking the Probability for Imminent Hypoglycemia in Diabetes from Self-Monitoring Blood Glucose (SMBG) Data”, filed Sep. 30, 2019; Publication No. US-2020-0066410-A1, Feb. 27, 2020.
U.S. Utility patent application Ser. No. 13/394,091, entitled “Tracking the Probability for Imminent Hypoglycemia in Diabetes from Self-Monitoring Blood Glucose (SMBG) Data”, filed Mar. 2, 2012; U.S. Pat. No. 10,431,342, issued Oct. 1, 2019.
International Patent Application Serial No. PCT/US2010/047711, entitled “Tracking the Probability for Imminent Hypoglycemia in Diabetes from Self-Monitoring Blood Glucose (SMBG) Data”, filed Sep. 2, 2010; Publication No. WO 2011/028925, Mar. 10, 2011.
U.S. Utility patent application Ser. No. 16/546,335, entitled “System Coordinator and Modular Architecture for Open-Loop and Closed-Loop Control of Diabetes”, filed Aug. 21, 2019; Publication No. US-2019-0374137-A1, Dec. 12, 2019.
U.S. Utility patent application Ser. No. 13/322,943, entitled “System Coordinator and Modular Architecture for Open-Loop and Closed-Loop Control of Diabetes”, filed Nov. 29, 2011; U.S. Pat. No. 10,420,489, issued Sep. 24, 2019.
International Patent Application Serial No. PCT/US2010/036629, entitled “System Coordinator and Modular Architecture for Open-Loop and Closed-Loop Control of Diabetes”, filed May 28, 2010; Publication No. WO 2010/138848, Dec. 2, 2010.
U.S. Utility patent application Ser. No. 16/451,766, entitled “TRACKING CHANGES IN AVERAGE GLYCEMIA IN DIABETICS”, filed Jun. 25, 2019; Publication No. US-2019-0318801-A1, Oct. 17, 2019.
U.S. Utility patent application Ser. No. 14/769,638, entitled “METHOD AND SYSTEM FOR MODEL-BASED TRACKING OF CHANGES IN AVERAGE GLYCEMIA IN DIABETES”, filed Aug. 21, 2015; U.S. Pat. No. 10,332,615, issued Jun. 25, 2019.
International Patent Application Serial No. PCT/US2014/017754, entitled “METHOD AND SYSTEM FOR MODEL-BASED TRACKING OF CHANGES IN AVERAGE GLYCEMIA IN DIABETES”, filed Feb. 21, 2014; Publication No. WO 2014/130841, Aug. 28, 2014.
U.S. Utility patent application Ser. No. 16/322,679, entitled “COMPOSITIONS AND METHODS FOR HIGH-SENSITIVITY IMMUNOASSAYS”, filed Feb. 1, 2019; Publication No. US 2021-0373011 A1, Dec. 2, 2021.
International Patent Application Serial No. PCT/US2017/045745, entitled “COMPOSITIONS AND METHODS FOR HIGH-SENSITIVITY IMMUNOASSAYS”, filed Aug. 7, 2017; Publication No. WO 2018/027221, Feb. 8, 2018.
U.S. Utility patent application Ser. No. 16/126,879, entitled “Method, System and Computer Program Product for Evaluation of Insulin Sensitivity, Insulin/Carbohydrate Ratio, and Insulin Correction Factors in Diabetes from Self-Monitoring Data”, filed Sep. 10, 2018; Publication No. US-2019-0019571-A1, Jan. 17, 2019.
U.S. Utility patent application Ser. No. 12/665,149, entitled “Method, System and Computer Program Product for Evaluation of Insulin Sensitivity, Insulin/Carbohydrate Ratio, and Insulin Correction Factors in Diabetes from Self-Monitoring Data”, filed Dec. 17, 2009; Publication No. 2010/0198520, Aug. 5, 2010.
International Patent Application Serial No. PCT/US2008/069416, entitled “Method, System and Computer Program Product for Evaluation of Insulin Sensitivity, Insulin/Carbohydrate Ratio, and Insulin Correction Factors in Diabetes from Self-Monitoring Data”, filed Jul. 8, 2008; Publication No. WO 2009/009528, Jan. 15, 2009.
U.S. Utility patent application Ser. No. 15/580,935, entitled “INSULIN MONITORING AND DELIVERY SYSTEM AND METHOD FOR CGM BASED FAULT DETECTION AND MITIGATION VIA METABOLIC STATE TRACKING”, filed Dec. 8, 2017; U.S. Pat. No. 11,311,665, issued Apr. 26, 2022.
International Patent Application Serial No. PCT/US2016/036729, entitled “INSULIN MONITORING AND DELIVERY SYSTEM AND METHOD FOR CGM BASED FAULT DETECTION AND MITIGATION VIA METABOLIC STATE TRACKING”, filed Jun. 9, 2016; Publication No. WO 2016/201120, Dec. 15, 2016.
U.S. Utility patent application Ser. No. 15/580,915, entitled “System and Method for Tracking Changes in Average Glycemia in Diabetics”, filed Dec. 8, 2017; Publication No. US-2018-0313815-A1, Nov. 1, 2018.
International Patent Application Serial No. PCT/US2016/036481, entitled “System and Method for Tracking Changes in Average Glycemia in Diabetics”, filed Jun. 8, 2016; Publication No. WO2016200970, Dec. 15, 2016.
U.S. Utility patent application Ser. No. 14/902,731, entitled “SIMULATION OF ENDOGENOUS AND EXOGENOUS GLUCOSE/INSULIN/GLUCAGON INTERPLAY IN TYPE 1 DIABETIC PATIENTS”, filed Jan. 4, 2016; U.S. Pat. No. 10,169,544, issued Jan. 1, 2019.
International Patent Application Serial No. PCT/US2014/045393, entitled “SIMULATION OF ENDOGENOUS AND EXOGENOUS GLUCOSE/INSULIN/GLUCAGON INTERPLAY IN TYPE 1 DIABETIC PATIENTS”, filed Jul. 3, 2014; Publication No. WO2015003124, Jan. 8, 2015.
U.S. Utility patent application Ser. No. 14/799,329, entitled “ACCURACY OF CONTINUOUS GLUCOSE SENSORS”, filed Jul. 14, 2015; U.S. Pat. No. 10,194,850, issued Feb. 5, 2019.
U.S. Utility patent application Ser. No. 12/065,257, entitled “Accuracy of Continuous Glucose Sensors”, filed Feb. 28, 2008; Publication No. 2008/0314395, Dec. 25, 2008.
International Patent Application Serial No. PCT/US2006/033724, entitled “Method for Improvising Accuracy of Continuous Glucose Sensors and a Continuous Glucose Sensor Using the Same”, filed Aug. 29, 2006; Publication No. WO07027691, Mar. 8, 2007.
U.S. Utility patent application Ser. No. 14/419,375, entitled “COMPUTER SIMULATION FOR TESTING AND MONITORING OF TREATMENT STRATEGIES FOR STRESS HYPERGLYCEMIA”, filed Feb. 3, 2015; U.S. Pat. No. 10,438,700, issued Oct. 8, 2019.
International Patent Application Serial No. PCT/US2013/053664, entitled “COMPUTER SIMULATION FOR TESTING AND MONITORING OF TREATMENT STRATEGIES FOR STRESS HYPERGLYCEMIA”, filed Aug. 5, 2013; Publication No. WO 2014/022864, Feb. 6, 2014.
U.S. Utility patent application Ser. No. 14/241,383, entitled “Method, System and Computer Readable Medium for Adaptive Advisory Control of Diabetes”, filed Feb. 26, 2014; U.S. Pat. No. 11,024,429, issued Jun. 1, 2021.
International Patent Application Serial No. PCT/US2012/052422, entitled “Method, System and Computer Readable Medium for Adaptive Advisory Control of Diabetes”, filed Aug. 26, 2012; Publication No. WO 2013/032965, Mar. 7, 2013.
U.S. Utility patent application Ser. No. 14/128,811, entitled “Methods and Apparatus for Modular Power Management and Protection of Critical Services in Ambulatory Medical Devices”, filed Dec. 23, 2013; U.S. Pat. No. 9,430,022, issued Aug. 30, 2016.
International Patent Application Serial No. PCT/US2012/043883, entitled “Methods and Apparatus for Modular Power Management and Protection of Critical Services in Ambulatory Medical Devices”, filed Jun. 22, 2012; Publication No. WO 2012/178113, Dec. 27, 2012.
U.S. Utility patent application Ser. No. 29/467,039, entitled “Alarm Clock Display of Personal Blood Glucose Level”, filed Sep. 13, 2013.
International Patent Application Serial No. PCT/US2013/042745, entitled “INSULIN-PRAMLINTIDE COMPOSITIONS AND METHODS FOR MAKING AND USING THEM”, filed May 24, 2013; Publication No. WO 2013/177565, Nov. 28, 2013.
U.S. Utility patent application Ser. No. 13/637,359, entitled “METHOD, SYSTEM, AND COMPUTER PROGRAM PRODUCT FOR IMPROVING THE ACCURACY OF GLUCOSE SENSORS USING INSULIN DELIVERY OBSERVATION IN DIABETES”, filed Sep. 25, 2012; U.S. Pat. No. 9,398,869, issued Jul. 26, 2016.
International Patent Application Serial No. PCT/US2011/029793, entitled “METHOD, SYSTEM, AND COMPUTER PROGRAM PRODUCT FOR IMPROVING THE ACCURACY OF GLUCOSE SENSORS USING INSULIN DELIVERY OBSERVATION IN DIABETES”, filed Mar. 24, 2011; Publication No. WO 2011/119832, Sep. 29, 2011.
U.S. Utility patent application Ser. No. 13/393,647, entitled “System, Method and Computer Program Product for Adjustment of Insulin Delivery (AID) in Diabetes Using Nominal Open-Loop Profiles”, filed Mar. 1, 2012; Publication No. 2012/0245556, Sep. 27, 2012.
International Patent Application Serial No. PCT/US2010/047386, entitled “System, Method and Computer Program Product for Adjustment of Insulin Delivery (AID) in Diabetes Using Nominal Open-Loop Profiles”, filed Aug. 31, 2010; Publication No. WO 2011/028731, Mar. 10, 2011.
U.S. Utility patent application Ser. No. 13/131,467, entitled “Method, System, and Computer Program Product for Tracking of Blood Glucose Variability in Diabetes”, filed May 26, 2011; U.S. Pat. No. 9,317,657, issued Apr. 19, 2016.
WWW. International Patent Application Serial No. PCT/US2009/065725, entitled “Method, System, and Computer Program Product for Tracking of Blood Glucose Variability in Diabetes”, filed Nov. 24, 2009; Publication No. WO 2010/062898, Jun. 3, 2010.
U.S. Utility patent application Ser. No. 12/975,580, entitled “Method, System, and Computer Program Product for the Evaluation of Glycemic Control in Diabetes from Self-Monitoring Data”, filed Dec. 22, 2010; Publication No. 2012/0004512, Jan. 5, 2012.
U.S. Utility patent application Ser. No. 11/305,946, entitled “Method, System, and Computer Program Product for the Evaluation of Glycemic Control in Diabetes from Self-Monitoring Data”, filed Dec. 19, 2005; U.S. Pat. No. 7,874,985, issued Jan. 25, 2011.
U.S. Utility patent application Ser. No. 10/240,228, entitled “Method, System, and Computer Program Product for the Evaluation of Glycemic Control in Diabetes from Self-Monitoring Data”, filed Sep. 26, 2002; U.S. Pat. No. 7,025,425, issued Apr. 11, 2006.
International Patent Application Serial No. PCT/US2001/009884, entitled “Method, System, and Computer Program Product for the Evaluation of Glycemic Control in Diabetes”, filed Mar. 29, 2001; Publication No. WO 01/72208, Oct. 4, 2001.
U.S. Utility patent application Ser. No. 12/664,444, entitled “Method, System and Computer Simulation Environment for Testing of Monitoring and Control Strategies in Diabetes”, filed Dec. 14, 2009; U.S. Pat. No. 10,546,659, issued Jan. 28, 2020.
International Patent Application Serial No. PCT/US2008/067725, entitled “Method, System and Computer Simulation Environment for Testing of Monitoring and Control Strategies in Diabetes”, filed Jun. 20, 2008; Publication No. WO 2008/157781, Dec. 24, 2008.
U.S. Utility patent application Ser. No. 12/516,044, entitled “Method, System, and Computer Program Product for the Detection of Physical Activity by Changes in Heart Rate, Assessment of Fast Changing Metabolic States, and Applications of Closed and Open Control Loop in Diabetes”, filed May 22, 2009; U.S. Pat. No. 8,585,593, issued Nov. 19, 2013.
International Patent Application Serial No. PCT/US2007/085588, entitled “Method, System, and Computer Program Product for the Detection of Physical Activity by Changes in Heart Rate, Assessment of Fast Changing Metabolic States, and Applications of Closed and Open Control Loop in Diabetes”, filed Nov. 27, 2007; Publication No. WO2008/067284, Jun. 5, 2008.
U.S. Utility patent application Ser. No. 12/159,891, entitled “Method, System and Computer Program Product for Evaluation of Blood Glucose Variability in Diabetes from Self-Monitoring Data”, filed Jul. 2, 2008; U.S. Pat. No. 11,355,238, issued Jun. 7, 2022.
International Patent Application Serial No. PCT/US2007/000370, entitled “Method, System and Computer Program Product for Evaluation of Blood Glucose Variability in Diabetes from Self-Monitoring Data”, filed Jan. 5, 2007; Publication No. WO07081853, Jul. 19, 2007.
U.S. Utility patent application Ser. No. 11/943,226, entitled “Systems, Methods and Computer Program Codes for Recognition of Patterns of Hyperglycemia and Hypoglycemia, Increased Glucose Variability, and Ineffective Self-Monitoring in Diabetes”, filed Nov. 20, 2007; Publication No. 2008/0154513, Jun. 26, 2008.
U.S. Utility patent application Ser. No. 11/578,831, entitled “Method, System and Computer Program Product for Evaluating the Accuracy of Blood Glucose Monitoring Sensors/Devices”, filed Oct. 18, 2006; U.S. Pat. No. 7,815,569, issued Oct. 19, 2010.
International Patent Application Serial No. US2005/013792, entitled “Method, System and Computer Program Product for Evaluating the Accuracy of Blood Glucose Monitoring Sensors/Devices”, filed Apr. 21, 2005; Publication No. WO05106017, Nov. 10, 2005.
U.S. Utility patent application Ser. No. 10/592,883, entitled “Method, Apparatus, and Computer Program Product for Stochastic Psycho-physiological Assessment of Attentional Impairments”, filed Sep. 15, 2006; U.S. Pat. No. 7,761,144, issued Jul. 20, 2010.
International Patent Application Serial No. US2005/008908, entitled “Method, Apparatus, and Computer Program Product for Stochastic Psycho-physiological Assessment of Attentional Impairments”, filed Mar. 17, 2005; Publication No. 2005089431, Sep. 29, 2005.
U.S. Utility patent application Ser. No. 10/524,094, entitled “Method, System, And Computer Program Product For The Processing Of Self-Monitoring Blood Glucose (SMBG) Data To Enhance Diabetic Self-Management”, filed Feb. 9, 2005; U.S. Pat. No. 8,538,703, issued Sep. 17, 2013.
International Patent Application Serial No. PCT/US2003/025053, entitled “Managing and Processing Self-Monitoring Blood Glucose”, filed Aug. 8, 2003; Publication No. WO 2004/015539, Feb. 19, 2004.
International Patent Application Serial No. PCT/US2002/005676, entitled “METHOD AND APPARATUS FOR THE EARLY DIAGNOSIS OF SUBACUTE, POTENTIALLY CATASTROPHIC ILLNESS”, filed Feb. 27, 2002; Publication No. WO02/67776, Sep. 6, 2002.
U.S. Utility patent application Ser. No. 09/793,653, entitled “METHOD AND APPARATUS FOR THE EARLY DIAGNOSIS OF SUBACUTE, POTENTIALLY CATASTROPHIC ILLNESS”, filed Feb. 27, 2001; U.S. Pat. No. 6,804,551, issued Oct. 12, 2004.
U.S. Utility patent application Ser. No. 10/069,674, entitled “Method and Apparatus for Predicting the Risk of Hypoglycemia”, filed Feb. 22, 2002; U.S. Pat. No. 6,923,763, issued Aug. 2, 2005.
International Patent Application Serial No. US00/22886, entitled “METHOD AND APPARATUS FOR PREDICTING THE RISK OF HYPOGLYCEMIA”, filed Aug. 21, 2000; Publication No. WO01/13786, Mar. 1, 2001.
Rewers M. The changing face of the epidemiology of insulin-dependent diabetes mellitus (IDDM): research designs and models of disease causation. Ann Med 1991; 23:419-426. PMID:1930939.
Rewers M, LaPorte R E, King H, Tuomilehto J. Trends in the prevalence and incidence of diabetes: insulin-dependent diabetes mellitus in childhood. World Health Stat Q 1988; 41:179-189. PMID:3394708.
Fredheim S, Johannesen J, Johansen A, et al. Diabetic ketoacidosis at the onset of type 1 diabetes is associated with future HbA1c levels. Diabetologia. 2013; 56(5):995-1003. doi: 10.1007/s00125-013-2850-z.
Duca L M, Wang B, Rewers M, Rewers A. Diabetic Ketoacidosis at Diagnosis of Type 1 Diabetes Predicts Poor Long-term Glycemic Control. Diabetes Care. 2017 September; 40(9): 1249-1255. doi: 10.2337/dc17-0558. Epub 2017 Jun. 30. PMID: 28667128.
Ghetti S, Kuppermann N, Rewers A, Myers S R, Schunk J E, Stoner M J, Garro A, Quayle K S, Brown K M, Trainor J L, Tzimenatos L, DePiero A D, McManemy J K, Nigrovic L E, Kwok M Y, Perry C S 3rd, Olsen C S, Casper T C, Glaser N S; Pediatric Emergency Care Applied Research Network (PECARN) DKA FLUID Study Group. Cognitive Function Following Diabetic Ketoacidosis in Children With New-Onset or Previously Diagnosed Type 1 Diabetes. Diabetes Care. 2020 November; 43(11):2768-2775. doi: 10.2337/dc20-0187. Epub 2020 Sep. 22. PMID: 32962981; PMCID: PMC7576431.
Aye T, Mazaika P K, Mauras N, et al. Impact of Early Diabetic Ketoacidosis on the Developing Brain. Diabetes Care. 2019; 42(3):443-449. doi: 10.2337/dc18-1405.
Lundgren M, Jonsdottir B, Elding Larsson H, DiPiS study group. Effect of screening for type 1 diabetes on early metabolic control: the DiPiS study. Diabetologia. 2019 January; 62(1):53-57.
Jacobsen L M, Haller M J, Schatz D A. Understanding Pre-Type 1 Diabetes: The Key to Prevention. Front Endocrinol (Lausanne). 2018; 9:70. Published 2018 Mar. 6. doi: 10.3389/fendo.2018.00070.
Sharma A, Liu X, Hadley D, Hagopian W, Chen W M, Onengut-Gumuscu S, Törn C, Steck A K, Frohnert B I, Rewers M, Ziegler A G, Lernmark \dot{A}, Toppari J, Krischer J P, Akolkar B, Rich S S, She J X, TEDDY Study Group. Identification of non-HLA genes associated with development of islet autoimmunity and type 1 diabetes in the prospective TEDDY cohort. J Autoimmun. 2018 May; 89:90-100. PMID: 29310926. PMCID: PMC 5902429.
Steck A, Dong F, Rasmussen C, Bautista K, Sepulveda F, Baxter J, Yu L, Frohnert B, Rewers M, ASK Study Group., Rewers M, Barbour A, Bautista K, Baxter J, Felipe-Morales D, Dong F, Dormond-Brooks P, Driscoll K, Frohnert B, Rasmussen C G, Gesualdo P, Hoffman M, Karban R, Sepulveda F, Shorrosh H, Simmons K, Steck A, Taki I, Waugh K, Yu L, Liu E, Gallant M, McQueen R B, Norris J M. CGM Metrics Predict Imminent Progression to Type 1 Diabetes: Autoimmunity Screening for Kids (ASK) Study. Diabetes Care December 2021, dc210602; DOI: 10.2337/dc21-0602.
Steck A K, Dong F, Taki I, Hoffman M, Simmons K, Frohnert B I, Rewers M J. Continuous Glucose Monitoring Predicts Progression to Diabetes in Autoantibody Positive Children. J Clin Endocrinol Metab. 2019 Aug. 1; 104(8):3337-3344. doi: 10.1210/jc.2018-02196. PMID: 30844073; PMCID: PMC6589073.
Brown S A, Basu A, Kovatchev B P. Beyond HbA1c: using continuous glucose monitoring metrics to enhance interpretation of treatment effect and improve clinical decision-making. Diabet Med. 2019 June; 36(6):679-687.
Kovatchev B. Glycemic Variability: Risk Factors, Assessment, and Control. J Diabetes Sci Technol. 2019 July; 13(4):627-635.
Clarke, W. L., Anderson, S. M., Breton, M. D., Patek, S. D., Kashmer, L. & Kovatchev, B. P. Closed-Loop Artificial Pancreas Using Subcutaneous Glucose Sensing and Insulin Delivery and a Model Predictive Control Algorithm: The Virginia Experience. J Diabetes Sci Technol, 3, 1031-1038 (2009).
Bruttomesso, D., Farret, A., Costa, S., Marescotti, M. C., Vettore, M., Avogaro, A., Tiengo, A., Dalla Man, C., Place, J., Facchinetti, A., Guerra, S., Magni, L., De Nicolao, G., Cobelli, C., Renard, E. & Maran, A. Closed-Loop Artificial Pancreas Using Subcutaneous Glucose Sensing & Insulin Delivery, and a Model Predictive Control Algorithm: Preliminary Studies in Padova and Montpellier. J Diabetes Sci Technol, 3, 1014-1021 (2009).
Kovatchev, B. P., Cobelli, C., Renard, E., Anderson, S., Breton, M., Patek, S., Clarke, W., Bruttomesso, D., Maran, A., Silvana, C., Avogaro, A., Dalla Man, C., Facchinetti, A., Magni, L., De Nicolao, G., Place, J. & Farret, A. Multinational Study of Subcutaneous Model-Predictive Closed-Loop Control in Type 1 Diabetes Mellitus: Summary of the Results. J Diabetes Sci Technol, 4, 1374-1381 (2010).
Renard, E. M., Farret, A., Place, J., Cobelli, C., Kovatchev, B. P. & Breton, M. D. Closed-loop insulin delivery using subcutaneous infusion and glucose sensing, and equipped with a dedicated safety supervision algorithm, improves safety of glucose control in type 1 diabetes. Diabetologia, 53 (Suppl 1), S25(2010).
Breton, M. D., Farret, A., Bruttomesso, D., Anderson, S. M., Magni, L., Patek, S., Dalla Man, C. Place, J. Demartini, S., Del Favero, S., Toffanin, C., Karvetski, C. H., Dassau, E., Zisser, H., Doyle, F. J. 3rd., De Nicolao, G., Avogaro, A., Cobelli, C., Renard, E. & Kovatchev, B. P. on behalf of The International Artificial Pancreas (iAP) Study Group. Fully-integrated artificial pancreas in type 1 diabetes: modular closed-loop glucose control maintains near-normoglycemia. Diabetes, 61, 2230-2237 (2012).
Del Favero, S., Bruttomesso, D., Di Palma, F., Lanzola, G., Visentin, R., Filippi, A., Scotton, R., Toffanin, C., Messori, M., Scarpellini, S., Hynes, P. K., Kovatchev, B. P., DeVries, J. H., Renard, E., Magni, L., Avogaro, A., Cobelli, C. on behalf of the AP@home Consortium. First Use of Model Predictive Control in Outpatient Wearable Artificial Pancreas. Diabetes Care, 37, 1212-1215 (2014).
Cobelli, C., Renard, E., Kovatchev, B. P., Keith-Hynes, P., Ben Brahim, N., Place, J., Del Favero, S., Breton, M. D., Farret, A., Bruttomesso, D., Dassau, E., Zisser, H., Doyle III, F. J., Patek, S. & Avogaro, A. Pilot Studies of Wearable Artificial Pancreas in Type 1 Diabetes. Diabetes Care, 35, e65-e67 (2012).
Kovatchev, B. P., Renard, E., Cobelli, C., Zisser, H., Keith-Hynes, P., Anderson, S. M. Brown, S. A. Chernavvsky, D. R., Breton, M. D., Farret, A., Pelletier, M. J., Place, J., Bruttomesso, D., Del Favero, S., Visentin, R., Filippi, A., Scotton, R., Avogaro, A. & Doyle III, F. J., Feasibility of Outpatient Fully Integrated Closed-Loop Control: First Studies of Wearable Artificial Pancreas. Diabetes Care, 36, 1851-1858 (2013).
Kovatchev, B. P., Renard, E., Cobelli, C., Zisser, H., Keith-Hynes, P., Anderson, S. M., Brown, S. A., Chernavvsky, D. R., Breton, M. D., Mize, L. B., Farret, A., Place, J., Bruttomesso, D., Del Favero, S., Boscari, F., Galasso, S., Avogaro, A., Magni, L., Di Palma, F., Toffanin, C., Messori, M., Dassay E. & Doyle III, F. Safety of Outpatient Closed-Loop Control: First Randomized Crossover Trials of a Wearable Artificial Pancreas. Diabetes Care, 37,1-8, DOI: 10.2337/dc13-2076 (2014).
Shepard, J. A., Gonder-Frederick, L. A., Vajda, K. & Kovatchev, B. P. Patient Perspectives on Personalized Glucose Advisory Systems for Type 1 Diabetes Management. Diabetes TechnolTher, 14, 858-861 (2012).
Chernavvsky, D., DeBoer, M. & Breton, M. D. Missed meal boluses: Closed-loop control minimizes hyperglycemia among adolescents with type 1 diabetes. ATTD Data Club Session, Vienna, (2014).
Lobo B, Farhy L, Shafiei M, and Kovatchev B. A data-driven approach to classifying continuous glucose monitoring (CGM) time series. IEEE Transactions on Biomedical Engineering, 2021. https://doi.org/10.1109/TBME.2021.3103127. PMID: 34375274.
Montaser E, Villard O, Fabris C, Lobo B, Brown S, DeBoer M, Kovatchev B, Farhy L. Detecting the Risk of Type 1 Diabetes Through Clustering of Data Collected During a Self-Administered CGM-based Home Test. Submitted to ATTD 2022.
Lobo B and Kovatchev B. External validation of a classifier of daily continuous glucose monitoring (CGM) profiles. Computers in Biology and Medicine, In Press, 2022.
Rich S S. Mapping genes in diabetes. Genetic epidemiological perspective. Diabetes. 1990 November; 39(11):1315-1319. PMID: 2227105.
Erlich H, Valdes A M, Noble J, Carlson J A, Varney M, Concannon P, Mychaleckyj J C, Todd J A, Bonella P, Fear A L, Lavant E, Louey A, Moonsamy P; Type 1 Diabetes Genetics Consortium. HLA DR-DQ haplotypes and genotypes and type 1 diabetes risk: Analysis of the Type 1 Diabetes Genetics Consortium families. Diabetes. 2008 April; 57(4):1084-1092. PMDI: 18252895.
Concannon P, Erlich H A, Julier C, Morahan G, Nerup J, Pociot F, Todd J A, Rich S S; Type 1 Diabetes Genetics Consortium. Type 1 diabetes: evidence for susceptibility loci from four genome-wide linkage scans in 1,435 multiplex families. Diabetes. 2005 October; 54(10):2995-3001. PMID: 16186404.
I Concannon P, Onengut-Gumuscu S, Todd J A, Smyth D J, Pociot F, Bergholdt R, Akolkar B, Erlich H A, Hilner J E, Julier C, Morahan G, Nerup J, Nierras C R, Chen W M, Rich S S; Type 1 Diabetes Genetics Consortium. Diabetes. 2008 October; 57(10):2858-2861. PMID: 18647951.
Concannon P, Chen W M, Julier C, Morahan G, Akolkar B, Erlich H A, Hilner J E, Nerup J, Nierras C, Pociot F, Todd J A, Rich S S; Type 1 Diabetes Genetics Consortium. Diabetes. 2009 April; 58(4):1018-1022. PMID: 19136655.
Barrett J C, Clayton D G, Concannon P, Akolkar B, Cooper J D, Erlich H A, Julier C, Morahan G, Nerup J, Nierras C, Plagnol V, Pociot F, Schulienburg H, Smth D J, Stevens H, Todd J A, Walker N M, Rich S S; T1DGC. Genome-wide association study and meta-analysis find that over 40 loci affect risk of type 1 diabetes. Nat Genet. 2009 June; 41(6):703-7. PMID: 19430480.
Cooper J D, Howson J M, Smyth D, Walker N M, Stevens H, Yang J H, She J X, Eisenbarth G S, Rewers M, Todd J A, Akolkar B, Concannon P, Erlich H A, Julier C, Morahan G, Nerup J, Nierras C, Pociot F, Rich S S; Type 1 Diabetes Genetics Consortium. Confirmation of novel type 1 diabetes risk loci in families. Diabetologia. 2012 April; 55(4):996-1000. PMID: 22278338.
Howson J M, Cooper J D, Smyth D J, Walker N M, Stevens H, She J X, Eisenbarth G S, Rewers M, Todd J A, Akolkar B, Concannon P, Erlich H A, Julier C, Morahan G, Nerup J, Nierras C, Pociot F, Rich S S; Type 1 Diabetes Genetics Consortium. Evidence of gene-gene and age-at-diagnosis effects in type 1 diabetes. Diabetes. 2012 November; 61(11):3012-3017. PMID: 22891215.
Evangelou M, Smyth D J, Fortune M D, Burren O S, Walker N M, Guo H, Onengut-Gumuscu S, Chen W M, Concannon P, Rich S S, Todd J A, Wallace C. A method for gene-based pathway analysis using genomewide association study summary statistics reveals nine new type 1 diabetes associations. Genet Epidemiol. 2014 December; 38(8):661-670. PMID: 25371288.
Onengut-Gumuscu S, Chen W M, Burren O, Cooper N J, Quinlan A R, Mychaleckyj J C, Farber E, Bonnie J K, Szpak M, Scholfield E, Achuthan P, Guo H, Fortune M D, Stevens H, Walker N M, Ward L D, Kundaje A, Kellis M, Daly M J, Barrett J C, Cooper J D, Deloukas P; Type 1 Diabetes Genetics Consortium, Todd J A, Wallace C, Concannon P, Rich S S. Fine mapping of type 1 diabetes susceptibility loci and evidence for colocalization of causal variants with lymphoid enhancers. Nat Genet. 2015 April; 47(4):381-386. PMID: 25751624.
Hu X, Deutsch A J, Lenz T L, Onengut-Gumuscu S, Han B, Chen W M, Howson J M, Todd J A, de Bakker P I, Rich S S, Raychaudhuri S. Additive and interaction effects at three amino acid positions in HLA-DQ and HLA-DR molecules drive type 1 diabetes. Risk. Nat Genet. 2015 August; 47(8):898-905. PMID: 26168013.
Chiou J, Geusz R J, Okino M L, Han J Y, Miller M, Melton R, Beebe E, Benaglio P, Huang S, Korgaonkar K, Heller S, Kleger A, Preissl S, Gorkin D U, Sander M, Gaulton K J. Interpreting type 1 diabetes risk with genetics and single-cell epigenomics. Nature. 2021 June; 594(7863):398-402. PMID: 34012112.
Onengut-Gumuscu S, Chen W M, Robertson C C, Bonnie J K, Farber E, Zhu Z, Oksenberg J R, Brant S R, Bridges S L Jr, Edberg J C, Kimberly R P, Gregersen P K, Rewers MJ, Steck A K, Black M H, Dabelea D, Pihoker C, Atkinson M A, Wagenknecht L E, Divers J, Bell R A; SEARCH for Diabetes in Youth; Type 1 Diabetes Genetics Consortium, Erlich H A, Concannon P, Rich S S. Type 1 diabetes risk in African-ancestry participants and utility of an ancestry-specific genetic risk score. Diabetes Care. 2019 March; 42(3):406-415. PMID: 30659077.
Gabriel S B, Schaffner S F, Nguyen H, Moore J M, Roy J, Blumenstiel B, Higgins J, DeFelice M, Lochner A, Faggart M, Liu-Cordero S N, Rotimi C, Adeyemo A, Cooper R, Ward R, Lander E S, Daly M J, Altshuler D. The structure of haplotype blocks in the human genome. Science. 2020 Jun. 21; 296(5576):2225-2229. PMID: 12029063.
Robertson C C, Inshaw J R J, Onengut-Gumuscu S, Chen W M, Santa Cruz D F, Yang H, Cutler A J, Crouch D J M, Farber E, Bridges S L Jr, Edberg J C, Kimberly R P, Buckner J H, Deloukas P, Divers J, Dabelea D, Lawrence J M, Marcovina S, Shah A S, Greenbaum C J, Atkinson M A, Gregersen P K, Oksenberg J R, Pociot F, Rewers M J, Steck A K, Dunger D B; Type 1 Diabetes Genetics Consortium, Wicker L S, Concannon P, Todd J A, Rich S S. Fine-mapping, trans-ancestral and genomic analyses identify causal variants, cells, genes and drug targets for type 1 diabetes. Nat Genet. 2021 July; 53(7):962-971. PMID: 34127860.
Oram R A, Patel K, Hill A, Bhields B, McDonald T J, Jones A, Hattersley A, Weedon M N. A type 1 diabetes genetic risk score can aid discrimination between type 1 and type 2 diabetes in young adults. Diabetes Care. 2016 March; 39(3):337-344. PMID: 26577414.
Sharp S A, Rich S S, Wood A R, Jones S E, Beaumont R N, Harrison J W, Schneider D A, Locke J M, Tyrrell J, Weedon M N, Hagopian W A, Oram R A. Development and standardization of an improved type 1 diabetes genetic risk score for use in newborn screening and incident diagnosis. Diabetes Care. 2019 February; 42(2):200-207. PMID: 30655379.
Onengut-Gumuscu S, Paila U, Chen W M, Ratan A, Zhu Z, Steck A K, Frohnert B I, Waugh K C, Webb-Robertson B J, Norris J M, Lange L A, Rewers M J, Rich S S. Novel genetic risk factors influence progression of islet autoimmunity to type 1 diabetes. Sci Rep. 2020 Nov. 5; 10(1):19193. PMID: 33154504.
Bezdek, J. C. (2013). Pattern recognition with fuzzy objective function algorithms. Springer Science & Business Media.
Rezaee, M. R., Lelieveldt, B. P., & Reiber, J. H. (1998). A new cluster validity index for the fuzzy c-mean. Pattern recognition letters, 19(3-4), 237-246.
Battelino T, Danne T, Bergenstal R M, Amiel S A, Beck R, Biester T, Bosi E, Buckingham B A, Cefalu W T, Close K L, Cobelli C, Dassau E, DeVries J H, Donaghue K C, Dovc K, Doyle F J 3rd, Garg S, Grunberger G, Heller S, Heinemann L, Hirsch I B, Hovorka R, Jia W, Kordonouri O, Kovatchev B, Kowalski A, Laffel L, Levine B, Mayorov A, Mathieu C, Murphy H R, Nimri R, Nørgaard K, Parkin C G, Renard E, Rodbard D, Saboo B, Schatz D, Stoner K, Urakami T, Weinzimer S A, Phillip M. Clinical Targets for Continuous Glucose Monitoring Data Interpretation: Recommendations From the International Consensus on Time in Range. Diabetes Care. 2019 August; 42(8):1593-1603. doi: 10.2337/dci19-0028. Epub 2019 Jun. 8. PMID: 31177185; PMCID: PMC6973648.
Cover, T., & Hart, P. (1967). Nearest neighbor pattern classification. IEEE transactions on information theory, 13(1), 21-27.
Bediaga N G, Li-Wai-Suen C S N, Haller M J, Gitelman S E, Evans-Molina C, Gottlieb P A P, Hippich M, Ziegler A G, Lernmark Å, DiMeglio L A, Wherrett D K, Colman P G, Harrison L C, Wentworth J M. Simplifying prediction of disease progression in pre-symptomatic type 1 diabetes using a single blood sample. Diabetologia 2021 November; 64(11):2432-2444. PMID: 34338806. PMCID: PMC8494707.
Ziegler A G, Kick K, Bonifacio E, Haupt F, Hippich M, Dunstheimer D, Lang M, Laub O, Warncke K, Lange K, Assfalg R, Jolink M, Winkler C, Achenbach P, Fr1da Study Group. Yield of a public health screening of children for islet autoantibodies in Bavaria, Germany. JAMA. 202 January 28; 323(4):339-351. PMID: 31990315. PMCID: PMC6990943.
Winkler C, Haupt F, Heigermoser M, Zapardiel-Gonzalo J, Ohli J, Faure T, Kalideri E, Hommel A, Delivani P, Berner R, Kordonouri O, Roloff F, von dem Berge T, Lange K, Oltarzewski M, Glab R, Szypowska A, Snape M D, Vatish M, Todd J A, Larsson H E, Ramelius A, Kördel JÅ, Casteels K, Paulus J, Ziegler A G, Bonifacio E, GPPAD Study Group. Identification of infants with increased type 1 diabetes genetic risk for enrollment into primary prevention trials—GPPAD-02 study design and first results. Pediatr Diabetes. 2019 September; 20(6): 720-727. PMID: 31192505. PMCID: PMC6851563.
Insel R A, Dunne J L, Atkinson M A, et al.: Staging presymptomatic type 1 diabetes: a scientific statement of JDRF, the Endocrine Society, and the American Diabetes Association. Diabetes Care 2015; 38(10):1964-1974. https://doi.org/10.2337/dc15-1419.
Ziegler A G, Rewers M, Simell O, et al.: Seroconversion to multiple islet autoantibodies and risk of progression to diabetes in children. JAMA 2013; 309(23):2473-2479. https://doi.org/10.1001/jama.2013.6285.
Wherrett D K, Chiang J L, Delamater A M, et al.: Type 1 Diabetes TrialNet Study Group. Defining pathways for development of disease-modifying therapies in children with type 1 diabetes: a consensus report. Diabetes Care 2015; 38(10):1975-1985. https://doi.org/10.2337/dc15-1429.
Elding Larsson H, Vehik K, Bell R, et al.: TEDDY Study Group; SEARCH Study Group; Swediabkids Study Group; DPV Study Group; Finnish Diabetes Registry Study Group. Reduced prevalence of diabetic ketoacidosis at diagnosis of type 1 diabetes in young children participating in longitudinal follow-up. Diabetes Care 2011; 34(11):2347-2352. https://doi.org/10.2337/dc11-1026.
Hekkala A M, Ilonen J, Toppari J, Knip M, Veijola R. Ketoacidosis at diagnosis of type 1 diabetes: effect of prospective studies with newborn genetic screening and follow up of risk children. Pediatr Diabetes 2018; 19(2):314-319. https://doi.org/10.1111/pedi.12541.
Ghetti S, Kuppermann N, Rewers A, et al.: Cognitive function following diabetic ketoacidosis in children with new-onset or previously diagnosed type 1 diabetes. Diabetes Care 2022; 43(11):2768-2775. https://doi.org/10.2337/dc20-0187.
Fredheim S, Johannesen J, Johansen A, et al.: Danish Society for Diabetes in Childhood and Adolescence. Diabetic ketoacidosis at the onset of type 1 diabetes is associated with future HbA1c levels. Diabetologia 2013; 56(5):995-1003. https://doi.org/10.1007/s00125-013-2850-z.
Duca L M, Wang B, Rewers M, Rewers A. Diabetic ketoacidosis at diagnosis of type 1 diabetes predicts poor long-term glycemic control. Diabetes Care 2017; 40(9):1249-1255. https://doi.org/10.2337/dc17-0558.
Aye T, Mazaika P K, Mauras N, et al.: Diabetes Research in Children Network (DirecNet) Study Group. Impact of early diabetic ketoacidosis on the developing brain. Diabetes Care 2019; 42(3):443-449. https://doi.org/10.2337/dc18-1405.
Lundgren M, Jonsdottir B, Elding Larsson H. Effect of screening for type 1 diabetes on early metabolic control: the DiPiS study. Diabetologia 2019; 62(1):53-57. https://doi.org/10.1007/s00125-018-4706-z.
Jacobsen L M, Haller M J, Schatz D A. Understanding pre-type 1 diabetes: the key to prevention. Front Endocrinol (Lausanne) 2018; 9:70. https://doi.org/10.3389/fendo.2018.00070.
Greenbaum C J. A key to T1D prevention: screening and monitoring relatives as part of clinical care. Diabetes 2021; 70(5):1029-37. https://doi.org/10.2337/db20-1112.
Tillil H, Kobberling J. Age-corrected empirical genetic risk estimates for first degree relatives of IDDM patients. Diabetes 1987; 36(1):93-99. https://doi.org/10.2337/diab.36.1.93.
Sims E K, Besser R E J, Dayan C, et al.: Screening for type 1 diabetes in the general population: a status report and perspective. Diabetes 2022; 71(4):610-623. https://doi.org/10.2337/dbi20-0054.
Vigersky R, Shrivastav M. Role of continuous glucose monitoring for type 2 in diabetes management and research. J Diabetes Complications 2017; 31(1):280-287. https://doi.org/10.1016/j.jdiacomp.2016.10.007.
Zou C C, Liang L, Hong F, Zhao Z Y. Glucose metabolism disorder in obese children assessed by continuous glucose monitoring system. World J Pediatr 2008; 4(1):26-30. https://doi.org/10.1007/s12519-008-0005-y.
Ehrhardt N, Al Zaghal E. Behavior Modification in Prediabetes and Diabetes: Potential Use of Real-Time Continuous Glucose Monitoring. J Diabetes Sci Technol 2019; 13(2):271-275. https://doi.org/10.1177/1932296818790994.
Steck A K, Dong F, Taki I, et al.: Early hyperglycemia detected by continuous glucose monitoring in children at risk for type 1 diabetes. Diabetes Care 2014; 37(7):2031-2033. https://doi.org/10.2337/dc13-2965.
Steck A K, Dong F, Taki I, et al.: Continuous glucose monitoring predicts progression to diabetes in autoantibody positive children. J Clin Endocrinol Metab 2019; 104(8):3337-3344. https://doi.org/10.1210/jc.2018-02196.
Steck A K, Dong F, Geno Rasmussen C, et al.: CGM metrics predict imminent progression to type 1 diabetes: autoimmunity screening for kids (ASK) study. Diabetes Care 2022; 45(2):365-371. https://doi.org/10.2337/dc21-0602.
Madhu S V, Muduli S K, Avasthi R. Abnormal glycemic profiles by CGMS in obese first-degree relatives of type 2 diabetes mellitus patients. Diabetes Technol Ther 2013; 15(6):461-465. https://doi.org/10.1089/dia.2012.0333.
Chon S, Lee Y J, Fraterrigo G, et al.: Evaluation of glycemic variability in well-controlled type 2 diabetes mellitus. Diabetes Technol Ther 2013; 15(6):455-460. https://doi.org/10.1089/dia.2012.0315.
Wilson D M, Pietropaolo S L, Acevedo-Calado M, et al.: CGM Metrics Identify Dysglycemic States in Participants From the TrialNet Pathway to Prevention Study. Diabetes Care 2023; 46(3):526-534. https://doi.org/10.2337/dc22-1297.
Acciaroli G, Sparacino G, Hakaste L, et al.: Diabetes and prediabetes classification using glycemic variability indices from continuous glucose monitoring data. J Diabetes Sci Technol 2018; 12(1):105-113. https://doi.org/10.1177/1932296817710478.
Güemes A, Cappon G, Hernandez B, et al.: Predicting quality of overnight glycaemic control in type 1 diabetes using binary classifiers. IEEE J Biomed Health Inform 2020; 24(5):1439-1446. https://doi.org/10.1109/JBHI.2019.2938305.
Kovatchev B P. Metrics for glycaemic control—from HbA1c to continuous glucose monitoring. Nat Rev Endocrinol 2017; 13(7):425-436. https://doi.org/10.1038/nrendo.2017.3.
Hastie T, Tibshirani R, Friedman J H. The Elements of Statistical Learning: Data Mining, Inference, and Prediction, Springer: New York, NY, USA; 2009; 2:1-758. https://doi.org/10.1007/978-0-387-21606-5.
Sharma S. Applied multivariate techniques. John Wiley & Sons, Inc., New York, NY, USA; 1996. https://dl.acm.org/doi/10.5555/225519.
Liu Y, Rafkin L E, Matheson D, et al.: Type 1 Diabetes TrialNet Study Group. Use of self-collected capillary blood samples for islet autoantibody screening in relatives: a feasibility and acceptability study. Diabet Med 2017; 34(7):934-937. https://doi.org/10.1111/dme.13338.
Herold K C, Bundy B N, Long S A, et al.: Type 1 Diabetes TrialNet Study Group. An anti-CD3 antibody, teplizumab, in relatives at risk for type 1 diabetes. N Engl J Med 2019; 381(7):603-613. https://doi.org/10.1056/NEJMoa1902226.
Montaser E, Breton M D, Brown S A, DeBoer M D, Kovatchev B P, Farhy L S. Predicting Immunological Risk for Stage 1 and 2 T1D Prediabetes using a One-Week CGM Home Test, Nocturnal Glucose Increments, and Standardized Liquid Mixed Meal Breakfasts, with Classification Enhanced by Machine Learning. Diabetes Technology and Therapeutics (in press).
Perišić M M, Vladimir K, Karpov S, et al.: Polygenic Risk Score and Risk Factors for Gestational Diabetes. J Pers Med. 2022 Aug. 26; 12(9):1381.
van Doorn W P T M, Foreman Y D, Schaper N C, et al.: Machine learning-based glucose prediction with use of continuous glucose and physical activity monitoring data: The Maastricht Study. PLoS One. 2021 Jun. 24; 16(6):e0253125.
Hastie T, Tibshirani R, Friedman J H. The Elements of Statistical Learning: Data Mining, Inference, and Prediction, Springer: New York, NY, USA; 2009; 2:1-758. https://doi.org/10.1007/978-0-387-21606-5.
Sharma S. Applied multivariate techniques. John Wiley & Sons, Inc., New York, NY, USA; 1996.
Vigersky R, Shrivastav M. Role of continuous glucose monitoring for type 2 in diabetes management and research. J Diabetes Complications 2017; 31(1):280-287.
Zou C C, Liang L, Hong F, Zhao Z Y. Glucose metabolism disorder in obese children assessed by continuous glucose monitoring system. World J Pediatr 2008; 4(1):26-30.
Ehrhardt N, Al Zaghal E. Behavior Modification in Prediabetes and Diabetes: Potential Use of Real-Time Continuous Glucose Monitoring. J Diabetes Sci Technol 2019; 13(2):271-275.
Steck A K, Dong F, Taki I, et al.: Early hyperglycemia detected by continuous glucose monitoring in children at risk for type 1 diabetes. Diabetes Care 2014; 37(7):2031-2033.
Steck A K, Dong F, Taki I, et al.: Continuous glucose monitoring predicts progression to diabetes in autoantibody positive children. J Clin Endocrinol Metab 2019; 104(8):3337-3344.
Steck A K, Dong F, Geno Rasmussen C, et al.: CGM metrics predict imminent progression to type 1 diabetes: autoimmunity screening for kids (ASK) study. Diabetes Care 2022; 45(2):365-371.
Madhu S V, Muduli S K, Avasthi R. Abnormal glycemic profiles by CGMS in obese first-degree relatives of type 2 diabetes mellitus patients. Diabetes Technol Ther 2013; 15(6):461-465.
Chon S, Lee Y J, Fraterrigo G, et al.: Evaluation of glycemic variability in well-controlled type 2 diabetes mellitus. Diabetes Technol Ther 2013; 15(6):455-460.
Wilson D M, Pietropaolo S L, Acevedo-Calado M, et al.: CGM Metrics Identify Dysglycemic States in Participants From the TrialNet Pathway to Prevention Study. Diabetes Care 2023; 46(3):526-534.

What is claimed is:

1. A database management system for efficient physiological diagnosis of a specified physiological disorder, the system comprising:
a physical data store containing glucose measurement data and a representation for at least one classification of the glucose measurement data, wherein:
the glucose measurement data is associated with at least one controlled glycemic-response consumption activity; and
the representation is an indication that a glucose measurement trace from the glucose measurement data is associated with a specified physiological disorder or is indicative of the specified physiological disorder; and
a processor and computer memory configured with instructions stored thereon that when executed will cause the processor to:
receive a new glucose measurement possibly associated with controlled glycemic-response consumption activity; and
classify the newly received glucose measurement trace by either evaluating the trace by a predefined feature-based classifier or by searching the physical data store by comparing a newly received glucose measurement trace to the at least one classification using a similarity metric and classifying the newly received glucose measurement trace with the representation based on a matched similarity metric in response to the comparing; and
ascribe a clinical recommendation or assessment based on the
representation of the classified newly received glucose measurement trace.

2. The system of claim 1, wherein:
the clinical recommendation or assessment includes a treatment related to initiating, modifying, or forgoing administration of synthetic insulin based on the representation.

3. The system of claim 1, wherein:
the controlled glycemic-response consumption activity includes consumption of an energy drink in lieu of a meal.

4. The system of claim 1, wherein:
the physical data store contains glucose measurement data from one or more of:
individuals that have low risk of the specified physiological disorder and not diagnosed with the specified physiological disorder;
individuals that have high risk of the specified physiological disorder and not diagnosed with the specified physiological disorder; or
individuals diagnosed with the specified physiological disorder;
the newly received glucose measurement trace is from:
an individual that has low risk of the specified physiological disorder and not diagnosed with the specified physiological disorder;
an individual that has high risk of the specified physiological disorder and not diagnosed with the specified physiological disorder; or
an individual diagnosed with the specified physiological disorder.

5. The system of claim 1, wherein:
the specified physiological disorder is diabetes; and
the autoantibody is an islet antibody.

6. The system of claim 1, wherein:
the representation is an estimate that the glucose measurement trace is classified as autoantibody positive or autoantibody negative; and
the similarity metric is a score that is a probability that the newly received glucose measurement trace is classified as autoantibody positive or autoantibody negative.

7. The system of claim 1 in combination with a glucose measurement device, wherein:
the glucose measurement device is configured to generate the glucose measurement for the newly received glucose measurement trace; and
the glucose measurement device is in communication with the processor or in communication with a data store that is in communication with the processor.

8. The system of claim 1, wherein:
the glucose measurement data contained in the physical data store is continuous glucose measurement data; and
the glucose measurements of the newly received glucose measurement trace is continuous glucose measurement data.

9. The system of claim 1, wherein:
the glucose measurement trace of the glucose measurement data contained in the physical data store is a compilation of glucose measurements taken for seven days; and
the newly received glucose measurement trace is a compilation of glucose measurements taken for seven days.

10. The system of claim 1, wherein instructions cause the processor to one or more of:
store the classification of the newly received glucose measurement trace in a data store that is in communication with one or more of a predictive modeling system, a decision support system, an insulin delivery system, an insulin monitoring system, or an automated control system configured to use the classification or representation as input;
transmit the classification of the newly received glucose measurement trace to one or more of a predictive modeling system, a decision support system, an insulin delivery system, an insulin monitoring system, or an automated control system configured to use the classification or representation as input; or
monitor, analyze, or influence a concentration of glucose levels in a fluid using the classification of the newly received glucose measurement trace.

11. The system of claim 1, wherein:
the classification of the glucose measurement data contained in the physical data store is based on one or more classifier models including a linear discriminant analysis (LDA) technique, a linear support vector machine (SVM) technique, a logistic regression (LR) technique, or a K-nearest neighbors (KNN) technique; and
the one or more classifier models generates or extracts one or more glucose measurement metric from the glucose measurement data, the one or more glucose measurement metric including:
a mean glucose (MG) value;
a percent of time in range glucose value;
a coefficient of variation (CV) glucose value;
a standard deviation (SD) glucose value;
a glucose range;
a low blood glucose index (LBGI) for hypoglycemia;
a high blood glucose index (HBGI) for hypoerglycemia;
an average daily risk range (ADRR) for hypo- and hyperglycemia; and/or
an overnight incremental area under the curve (IAUC) value.

12. The system of claim 11, wherein:
the percent of time in range glucose value is based on glucose measurements falling within one or more time ranges.

13. The physiological diagnosis system of claim 12, wherein:
the one or more time ranges includes:
percentage of time glucose measurements are >180 mg/dL (T180);
percentage of time glucose measurements are >160 mg/dL (T160);
percentage of time glucose measurements are >140 mg/dL (T140); and
percentage of time glucose measurements are <70 mg/dL (T70); and
percentage of time glucose measurements are <54 mg/dL (T54).

14. The system of claim 1, wherein:
the physical data store includes c-peptide data associated with the controlled glycemic-response consumption activity, wherein the classification is further based on the c-peptide data.

15. The system of claim 14, wherein:
the instructions cause the processor to receive c-peptide measurements to generate newly received c-peptide data.

16. The system of claim 15 in combination with a c-peptide measurement device, wherein:
the c-peptide measurement device is configured to generate the newly received c-peptide data;
the c-peptide device measurement device is in communication with the processor or in communication with a data store that is in communication with the processor.

17. A method for managing a database for efficient physiological diagnosis of a specified physiological disorder, the method comprising:
receiving a glucose measurement possibly associated with controlled glycemic-response consumption activity;
classifying a newly received glucose measurement trace by either evaluating the trace by a predefined feature-based classifier or by searching a physical data store by comparing the newly received glucose measurement trace to at least one classification using a similarity metric and classifying the newly received glucose measurement trace with the representation based on a matched similarity metric in response to the comparing, the physical data store containing glucose measurement data and a representation for the at least one classification of the glucose measurement data, wherein:
the glucose measurement data is associated with at least one controlled glycemic-response consumption activity; and
the representation is an indication that a glucose measurement trace from the glucose measurement data is associated with a specified physiological disorder or is indicative of the specified physiological disorder; and
ascribing a clinical recommendation or assessment based on the representation of the classified newly received glucose measurement trace.

18. The method of claim 17, wherein:
the clinical recommendation or assessment includes a treatment related to initiating, modifying, or forgoing administration of synthetic insulin based on the representation.

19. The method of claim 17, wherein:
the controlled glycemic-response consumption activity includes consumption of the energy drink in lieu of a meal.

20. The method of claim 17, wherein:
the physical data store contains glucose measurement data from one or more of:
individuals that have low risk of the specified physiological disorder and not diagnosed with the specified physiological disorder;
individuals that have high risk of the specified physiological disorder and not diagnosed with the specified physiological disorder; or
individuals diagnosed with the specified physiological disorder;
the newly received glucose measurement trace is from:
an individual that has low risk of the specified physiological disorder and not diagnosed with the specified physiological disorder;
an individual that has high risk of the specified physiological disorder and not diagnosed with the specified physiological disorder; or
an individual diagnosed with the specified physiological disorder.
